(19) United States
(12) Patent Application Publication
(10) Pub. No.: US 2019/0017060 A1
(43) Pub. Date: Jan. 17, 2019

(54) MANIPULATION OF PROANTHOCYANIDIN (PA) COMPOSITION BY AFFECTING ANTHOCYANIDIN SYNTHASE (ANS) AND LEUCOANTHOCYANIDIN DIOXYGENASE (LDOX)

(71) Applicant: University of North Texas, Denton, TX (US)

(72) Inventors: Richard A. Dixon, Sulphur, OK (US); Ji Hyung Jun, Denton, TX (US)

(73) Assignee: University of North Texas, Denton, TX (US)

(21) Appl. No.: 16/037,042

(22) Filed: Jul. 17, 2018

Related U.S. Application Data
(60) Provisional application No. 62/533,356, filed on Jul. 17, 2017.

(51) Int. Cl. C12N 15/82

(52) U.S. Cl. CPC C12N 15/8243

(57) ABSTRACT

Using methods described in preferred embodiments, the composition of proanthocyanidins (PAs) in plants can be modified to produce plants that produce PAs with specific selections of catechin or epicatechin starter units, as well as specific selections of catechin or epicatechin extension units. Reduction or elimination of expression of the leucoanthocyanidin dioxygenase (ldox) gene or the anthocyanidin synthase (ans) gene, or both, is implemented in preferred embodiments to produce modified plants having modified PA content.

### BACKGROUND

This application claims priority to U.S. Provisional Patent Application No. 62/533,356, entitled “Manipulation of Proanthocyanidin (PA) Composition by Affecting Anthocyanidin Synthase (ANS) and Leucoanthocyanidin Dioxygenase (LDOX),” filed Jul. 17, 2017, the entire contents of which are hereby incorporated by reference.

Proanthocyanidins (PAs) are the second most abundant polyphenolic compounds found in a variety of plants. PAs are currently attracting attention due to their medicinal and nutritional values resulting from their antioxidant and organoleptic properties and are generally considered to act in defense mechanisms. The presence of PAs has been also considered as an important trait in forage crops to prevent pasture bloat and improve nitrogen nutrition in ruminant livestock as well as enhance soil nitrogen retention.

Plants contain 2,3-trans flavan-3-ols, i.e. (+)-(gallo)catechin and 2,3-cis flavan-3-ols, i.e. (−)-epi(gallo)catechin. A series of transcriptional regulators, key enzymes (anthocyanidin reductase (ANR) for production of epicatechin and leucoanthocyanidin reductase (LAR) for production of catechin) and transporters involved in the PA biosynthetic pathway have been identified through genetic and biochemical studies. So far, detailed information on PA compositions regarding terminal and extension units is limited, and the mechanism to determine the composition of PAs and the enzymatic or non-enzymatic reactions leading to PA condensation in vivo remain unclear. However, some data indicate that PA polymerization is not a random incorporation process of flavan-3-ol monomers. In grape varieties, trans subunits are more abundant as terminal units (˜97%) while the major extension units in the PAs were found in the cis-configuration (˜93%). A recent study shows that different ratios between cis- and trans-subunits in terminal and extension units leads to different sizes of PAs in different tissues. It has also been reported that oxidative degradation of PAs is different in tissues with different PA compositions.

In the seed coats of some leguminous plants (e.g. Medicago and soybean), PAs are exclusively composed of epicatechin, although the plants possess both ANR and LAR expression indicating that unknown mechanisms must exist to control the specific composition of PAs in such plants. In the seeds, PAs are known to contribute to seed coat color and integrity and it is likely that a large proportion of the PAs is oxidized into brown complexes which are strongly bound to the cell wall. Thus, solubility, cell wall binding property or efficiency of enzymatic or non-enzymatic oxidation can be affected by the composition of PAs. In fact, high accumulation of PAs in vegetative tissues of plants such as Arabidopsis and alfalfa, which naturally produce epicatechin-based PAs only in their seeds, is known to have adverse effects on growth and development of transgenic plants. In contrast, (+)-catechin is abundantly found in the soluble PAs of plants which naturally accumulate high levels of PAs in foliar tissue (e.g. Desmodium and Lotus). Thus, the modification of PA composition can be used to make forage plants such as alfalfa produce high concentration of PAs with reduced harmful effect. The modification of composition can be utilized to increase the soluble (extractable) PAs in the juice for nutritional benefits or to adjust the astringency and taste of beverages and wine. Also, the change of PA composition in relation to the process of oxidation can be used to improve management of oxidation during wine production and storage. Finally, being able to easily generate PAs with different and discrete compositions will provide evidence as to which types of PAs are best able to protect animals against pasture bloat and protect rumenal nitrogen.

### SUMMARY

Plants possess proanthocyanidins (PAs) of varying composition mainly composed of (−)-epicatechin and/or (+)-catechin. As shown in FIG. 1, flavan-3-ols are synthesized through the flavonoid pathway, sharing early biosynthetic steps with anthocyanin. In FIG. 1, the classes of compounds are indicated, with structures illustrating the precursors of catechin and epicatechin. Abbreviations for enzymes are: CHS, chalcone synthase; CHI, chalcone isomerase; F3H, flavanone 3-hydroxylase; DFR, dihydroflavonol reductase; ANS, anthocyanidin synthase; LDOX, leucoanthocyanidin dioxygenase; LAR, leucoanthocyanidin reductase; ANR, anthocyanidin reductase; UGT, UDP-glucosyltransferase. Leucoanthocyanidin is converted to anthocyanidin by anthocyanidin synthase (ANS) followed by anthocyanidin reductase (ANR) to generate (−)-epicatechin. Alternatively, leucoanthocyanidin is converted to (+)-catechin by leucoanthocyanidin reductase (LAR). The proteins involved in trafficking of PA monomers into the vacuole, and their polymerization have been reported. However, the exact mechanism by which the flavan-3-ol monomers are assembled to produce PAs with specific compositions in specific plants is still unknown.

The present disclosure demonstrates that expression of an ANS homologous protein (named LDOX) in Medicago truncatula is responsible for the exclusive (−)-epicatechin composition of Medicago PA polymers although (+)-catechin monomer can be been detected in the early stage of seed development. Both ANS and LDOX can convert (+)-catechin generated by LAR to cyanidin which can be reduced by ANR to (−)-epicatechin. However, mutations in ans and ldox affect the nature of the extension and starter units, respectively, suggesting that ANS activity in vivo is mainly involved in the generation of extension unit derived from leucocyanidin and LDOX is involved in providing initiator of PA polymer originating from catechin formed from leucocyanidin by LAR activity. Furthermore, the ans ldox double mutant produces (+)-catechin based PAs resulting in increase of soluble PAs and decrease of insoluble PAs compared to wild type.

The data confirm that loss of ANS and/or LDOX functionality changes PA composition and solubility. This is demonstrated particularly with regard to Medicago truncatula, a model legume that possesses both ANS and LDOX genes. Adjusting the regulation of ANS and/or LDOX functionality is expected to have the same effects in any plant that expresses ANS and/or LDOX and particularly in plants known to polymerize PAs in a manner that is affected by any of these genes. These include the economically important alfalfa, clover, soybean, grape, cacao, tea or strawberry plants.

### BRIEF DESCRIPTION OF THE DRAWINGS

FIG. 1 shows a scheme of the flavonoid pathway leading to proanthocyandin (PA) production.

FIG. 2A shows a schematic of the ANS gene depicting Tnt1 insertion positions in ans-1 (NF20424) and ans-2 (NF10529).

FIG. 2B shows a schematic of the LDOX gene depicting Tnt1 insertion positions in ldox-1 (NF11718) and ldox-2 (NF20282).

FIG. 2C shows RT-PCR for detecting full-length ANS and LDOX transcripts in wild-type (R108), ans-1, ans-2, ldox-1 and ldox-2.

FIG. 3A shows anthocyanin accumulation in wild type (R108), ans-1, ans-2, ldox-1 and ldox-2 7-day old seedlings.

FIG. 3B shows anthocyanin levels in wild type (R108), ans-1, and ldox-1 mutants.

FIG. 4A shows soluble PA levels in wild type (R108), ans-1, ans-2, ldox-1 and ldox-2 4-DAP pods as quantified with DMACA reagent and expressed as epicatechin equivalents.

FIG. 4B shows insoluble PA levels in wild type (R108), ans-1, ans-2, ldox-1 and ldox-2 4-DAP pods as quantified by the butanol-HCl method and expressed as procyanidin B1 equivalents.

FIG. 4C shows seed coat phenotypes of wild type (R108), ans-1 and ldox-1 dry seeds.

FIG. 5A shows extracted ion chromatogram (EIC) of epicatechin and catechinin monomers (m/z 289) in soluble PAs extracted from wild type (R108), ans and ldox 4-DAP pods.

FIG. 5B shows mass spectra of peaks in FIG. 5A.

FIG. 5C shows EIC of procyanidin dimers (m/z 577) in soluble PAs extracted from wild type (R108), ans and ldox 4-DAP pod.

FIG. 5D shows mass spectra of peaks in FIG. 5C.

FIG. 6A shows a scheme of phloroglucinolysis reaction.

FIG. 6B shows phloroglucinolysis of soluble PAs (50% methanol-wash fraction) from wild-type (R108), ans and ldox seeds.

FIG. 6C shows phloroglucinolysis of soluble PAs (50% acetone-elution fraction) from R108 (wild-type), ans and ldox seeds.

FIG. 7A shows HPLC profile of enzyme assay of ANS and LDOX with leucocyanidin at absorbance of 530 nm.

FIG. 7B shows HPLC profile of enzyme assay of ANS and LDOX with leucocyanidin at absorbance of 280 nm.

FIG. 7C shows a scheme of ANS and LDOX reaction with leucocyanidin.

FIG. 8A shows HPLC profiles of an enzyme reaction performed with (+)-catechin as substrate and recombinant ANS or LDOX protein at absorbance at 530 nm.

FIG. 8B shows HPLC profiles of an enzyme assay performed with (−)-catechin, (−)-epicatechin or (+)-epicatechin as substrate and purified recombinant ANS or LDOX protein at absorbance of 530 nm.

FIG. 8C shows a scheme of ANS and LDOX reaction with (+)-catechin.

FIG. 9A shows HPLC profiles at asbsorbance of 280 nm of an enzyme assay performed with (+)-catechin as substrate and recombinant ANS or LDOX only or combined with ANR protein.

FIG. 9B shows HPLC profiles with chiral column for the enzyme assay in FIG. 9A.

FIG. 9C shows a scheme of ANS and LDOX reactions combined with ANR with (+)-catechin as substrate.

FIG. 10A shows soluble PA levels in wild type (R108), ldox, lar and ldox lar 4-DAP pods as quantified with DMACA reagent and expressed as epicatechin equivalents.

FIG. 10B shows insoluble PA levels in wild type (R108), ldox, lar and ldox lar 4-DAP pods as quantified by the butanol-HCl method and expressed as procyanidin B1 equivalents.

FIG. 10C shows EIC of epicatechin and catechin monomers (m/z 289) in soluble PAs extracted from wild type (R108), ldox, lar and ldox lar 4-DAP pods.

FIG. 10D shows mass spectra of peaks in FIG. 10C.

FIG. 10E shows EIC of procyanidin dimers (m/z 577) in soluble PAs extracted from R108, ldox, lar and ldox lar 4-DAP pods.

FIG. 10F shows mass spectra of peaks in FIG. 10E.

FIG. 11A shows anthocyanin accumulation in wild type (R108), ans, ldox and ans ldox 7-days old seedlings.

FIG. 11B shows seed coat phenotypes of wild type (R108), ans, ldox and ans ldox dry seeds.

FIG. 11C shows soluble PA levels in wild type (R108), ans, ldox, and ans ldox 4-DAP pods, 16-DAP and dry seeds as quantified with DMACA reagent and expressed as epicatechin equivalents.

FIG. 11D shows soluble PA levels in wild type (R108), ans, ldox, and ans ldox 4-DAP pods, 16-DAP and dry seeds quantified by the butanol-HCl method and expressed as procyanidin B1 equivalents.

FIG. 12A shows EIC of epicatechin and catechin monomers (m/z 289) in soluble PAs extracted from R108, ans, ldox and ans ldox 4-DAP pods.

FIG. 12B shows mass spectra of peaks in FIG. 12A.

FIG. 12C shows EIC of procyanidin dimers (m/z 577) in soluble PAs extracted from R108, ans, ldox and ans ldox 4-DAP pods.

FIG. 12D shows mass spectra of peaks in FIG. 12C.

FIG. 13A shows phloroglucinolysis of soluble PAs (50% methanol-washing fraction) from R108 (wild-type), ans, ldox and ans ldox seeds.

FIG. 13B shows phloroglucinolysis of soluble PAs (50% acetone-elution fraction) from R108 (wild-type), ans, ldox and ans ldox seeds.

FIG. 14A shows a summary of PA biosynthesis for wild type (R108).

FIG. 14B shows a summary of PA biosynthesis for ans.

FIG. 14C shows a summary of PA biosynthesis for ldox.

FIG. 14D shows a summary of PA biosynthesis for ans ldox double mutants.

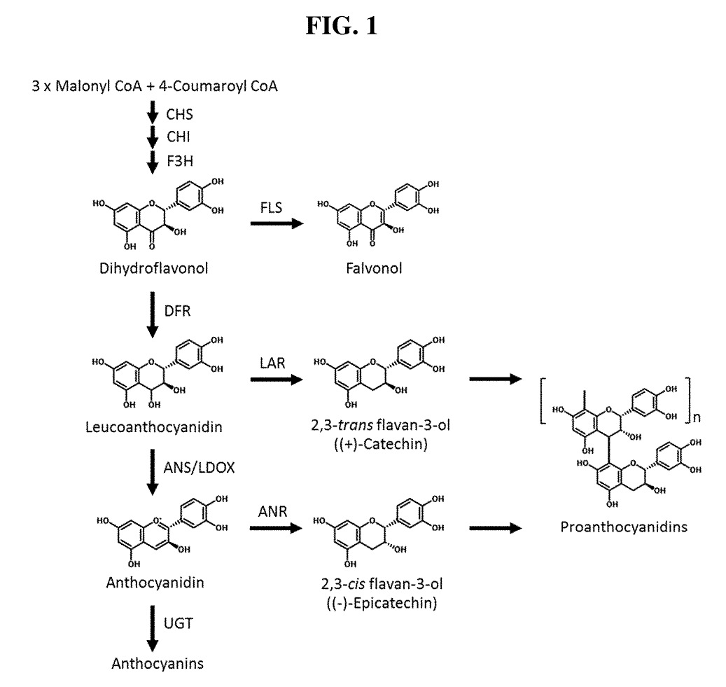

### DETAILED DESCRIPTION OF PREFERRED EMBODIMENTS

The present disclosure relates to manipulating, adjustment and/or control over the composition of proanthocyanidins (PAs) in plants. In particular, the present disclosure relates to modified plants, and methods for producing modified plants, that produce PAs having specific compositions. The modified plants produce PAs with specific selections of catechin or epicatechin starter units, as well as specific selections of catechin or epicatechin extension units.

One preferred embodiment relates to a method for producing a modified plant that has an increase in insoluble PA content, and a reduced astringency. In this preferred embodiment, the modified plant produces PAs having a catechin starter unit and epicatechin extension units. The modified plant has reduced or eliminated expression of the leucoanthocyanidin dioxygenase (ldox) gene and produces proanthocyanidin (PA) with a catechin starter unit and epicatechin extension units. The reduction or elimination of expression of the leucoanthocyanidin dioxygenase (ldox) gene is accomplished by introducing a mutation into a leucoanthocyanidin dioxygenase (ldox) gene in substantially all cells of the plant. “Substantially all cells” means that the mutation need not be present in all cells but should be present in a sufficient number to produce the desired change in PA composition with regard to the plant as a whole.

An additional preferred embodiment relates to a method for producing a modified plant that has an increase in soluble or extractable PA content. In this preferred embodiment, the modified plant produces PAs having an epicatechin starter unit and catechin extension units. The modified plant has reduced or eliminated expression of the anthocyanidin synthase (ans) gene and produces proanthocyanidin (PA) with an epicatechin starter unit and catechin extension units. The reduction or elimination of expression of the anthocyanidin synthase (ans) gene is accomplished by introducing a mutation into a anthocyanidin synthase (ans) gene in substantially all cells of the plant.

An additional preferred embodiment relates to a method for producing a modified plant that has an increase in soluble or extractable PA content. In this preferred embodiment, the modified plant produces PAs having a catechin starter unit and catechin extension units. The modified plant has reduced or eliminated expression of the leucoanthocyanidin dioxygenase (ldox) gene and the anthocyanidin synthase (ans) gene. The reduction or elimination of expression of the leucoanthocyanidin dioxygenase (ldox) gene and the anthocyanidin synthase (ans) gene is accomplished by introducing a mutation into both genes—both the leucoanthocyanidin dioxygenase (ldox) gene and the anthocyanidin synthase (ans) gene—in substantially all cells of the plant. Modified plants produced by this method have PAs that are more resistant to oxidation than those of unmodified plants, and PAs with reduced toxicity than those of unmodified plants.

Further preferred embodiments relate to the modified plants produced by the methods described above, as well as the seeds of the modified plant. In certain preferred embodiments the plant is a Medicago truncatula plant. In additional preferred embodiments the plant is alfalfa, clover, soybean, grape, cacao, tea or strawberry.

1. Identification of ans and ldox Mutants

Wild-type plants refer to Medicago truncatula ecotype R108. ans and ldox mutants were isolated by screening a tobacco Tnt1 transposon mutagenized Medicago R108 population as described by Tadege et al. ans-1 (NF20424), ans-2 (NF10529), ldox-1 (NF11718) and ldox-2 (NF20282) were obtained from The Samuel Roberts Noble Foundation, Ardmore, Okla. Seeds were scarified with concentrated sulfuric acid for 10 min, then washed with water five times to remove sulfuric acid. Scarified seeds were sterilized with 30% bleach for 10 min and then rinsed five times with sterile water. Sterilized seeds were vernalized at 4° C. for 4 days on B5 medium. Vernalized seeds were germinated on B5 medium for 10 days before transfer to soil in pots. The plants were grown in a growth chamber set at 16 h/8 h day/night cycle, 22° C.

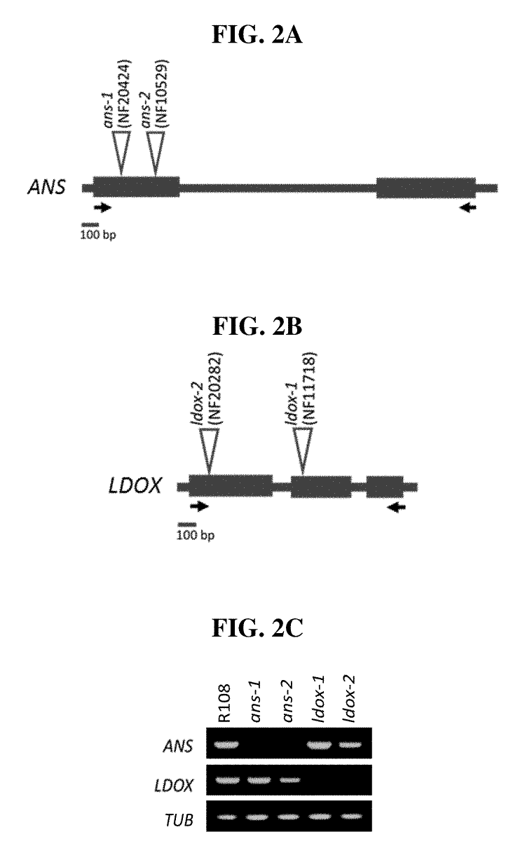

To understand the function of ANS (Medtr5g011250.1) and LDOX (Medtr3g072810.1), a Tnt1 transposon mutagenized population of Medicago was screened and two independent mutant alleles for each gene were obtained. ans-1 and ans-2 harboring Tnt1 insertions in the first exon of ANS and ldox-1 and ldox-2 with Tnt1 insertions in the first or second exon of the LDOX gene, respectively, were identified. FIG. 2A shows a schematic of the ANS gene depicting the Tnt1 insertion positions in ans-1 and ans-2. FIG. 2B shows a schematic of the LDOX gene depicting Tnt1 insertion positions in ldox-1 and ldox-2. Gene direction is from left to right. Solid boxes indicate exons and thinner lines indicate introns. The positions of primer pairs used for the PCR in FIG. 2C are shown by arrows. Tnt1 insertional mutants were confirmed by PCR with gene specific primers and a Tnt1 transposon specific primer. The following primers were used. For ans-1 and ans-2; Tnt1-F, ACAGTGCTACCTCCTCTGGATG (SEQ ID NO:1) and ANS-Tn-F, ATGGGAACGGTGGCTCAAAGA (SEQ ID NO:2). For ldox-1 and ldox-2; Tnt1-R, TGTAGCACCGAGATACGGTAATTAACAAGA (SEQ ID NO:3) and LDOX-Tn-F, GGAAGTCAAAAGAGTACAAACT (SEQ ID NO:4).

FIG. 2C shows RT-PCR for detecting full-length ANS and LDOX transcripts in wild-type (R108), ans-1 and ans-2, ldox-1 and ldox-2 where the PCR was run for 33 cycles. RNA was isolated from 12 DAP (days after pollination) seeds dissected from pods, using a Qiagen RNAeasy kit (Qiagen) according to the manufacturer's instructions. RNA was treated with DNase I to remove trace amounts of DNA contamination. One 1 μg of total RNA was used for reverse transcription with SuperScript® III Reverse Transcriptase (Thermo Fisher). For RT-PCR, primers ANS-F, ATGGGAACGGTGGCTCAAAGAGTTGA (SEQ ID NO:5); ANS-R, TCATTTTTTGGGATCATCTTTCTTCTCCT (SEQ ID NO:6); LDOX_F, ATGGAAGTCAAAAGAGTACAAACTTTAGCT (SEQ ID NO:7) and LDOX_R, CTACTGGGGAATCTTGTTGAATTTGC (SEQ ID NO:8) were used for amplification of full-length ANS and LDOX transcripts. The primers Tub-F, TTTGCTCCTCTTACATCCCGTG (SEQ ID NO:9) and Tub-R, GCCATGGAGAGACCTCTAGG, (SEQ ID NO:10) were used for tubulin gene amplification.

As seen in FIG. 2C, full-length transcripts of ANS or LDOX were not detectable in homozygous mutant plants indicating that the mutants are transcript null alleles.

2. Phenotypic Characterization of ans and ldox Mutants

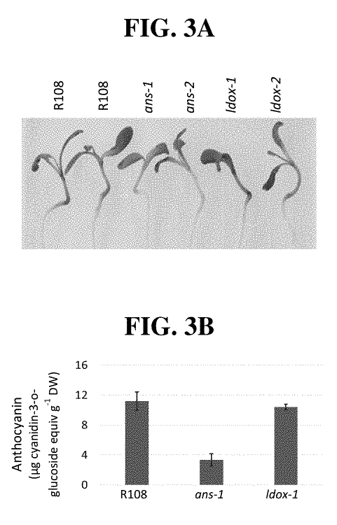

FIG. 3 shows the characterization of ans and ldox mutants in M. truncatula. As seen in FIG. 3A, ans mutants have no purple anthocyanin accumulation in the hypocotyl and junction of hypocotyl and root whereas ldox mutant seedlings accumulate anthocyanin normally compared to the wild type (R108) plants when plants were grown on B5 medium for 7 days. Anthocyanin level was measured using the same seedling plants. Plant materials were first ground to a powder in liquid nitrogen and extracted with 1 ml methanol: 0.1% HCl and anthocyanin measured as described by Pang et al. As seen in FIG. 3B, ans mutant plants accumulated only 30% of anthocyanin compared to R108 plants while ldox mutants did not show significant change of anthocyanin level. The data indicate that anthocyanin accumulation is mainly dependent on ANS activity at this stage of development. Error bars represent SD of three technical replicates.

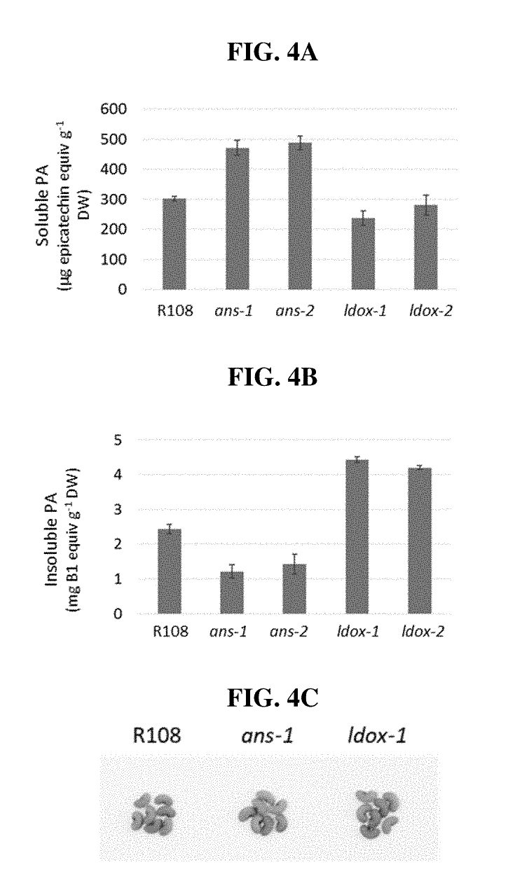

FIG. 4 shows phenotype of proanthocyanidin accumulation in ans and ldox mutants. In FIG. 4, PA content was measured as described by Pang et al with minor modifications. To check changes in PA accumulation, about 100 mg of samples were ground into powder in liquid nitrogen. The powder was extracted with 1.5 mL of proanthocyanidin extraction solvent (PES, 70% acetone with 0.5% acetic acid) by sonicating in a water bath for 30 min at room temperature. The resulting slurry was centrifuged at 5000 g for 5 min and supernatants were collected. The pellets were re-extracted twice, all supernatants were pooled, and pellets were saved for analysis of insoluble PAs. Equal volumes of chloroform were added to pooled supernatants and the mixtures vortexed for 30 s, centrifuged at 5000 g for 5 min, and the supernatants further extracted twice with chloroform and twice with hexane. The resulting aqueous phase (soluble PA fraction) was lyophilized and re-dissolved in 50% methanol. PAs in the soluble fraction were quantified by the DMACA method. Five μL of soluble PA fraction were mixed with 200 μL of 0.2% DMACA in methanol/HCl 1:1, and the OD at 640 nm was measured after 5 min. Epicatechin was used as standard.

Insoluble PA content was determined by the butanol/HCl method. The pellet after extraction with PES was lyophilized, 1 mL of butanol/HCl (95:5) was added, and the mixture was sonicated for 1 h to re-suspend the pellet, followed by heating at 95° C. for 1 h. The mixture was then allowed to cool, centrifuged at 12,000 g for 10 min, and the OD at 550 nm was measured. Procyanidin B2 was used as standard and processed in parallel with experimental samples.

Soluble PA (FIG. 4A) and insoluble PA (FIG. 4B) contents were measured with young pods containing developing seeds of wild type (R108), ans and ldox mutants at 4 days after pollination (DAP). Soluble PA contents were measured by the DMACA method and expressed as epicatechin equivalents. Insoluble PA contents were measured by the butanol/HCl method and expressed as procyanidin B2 equivalents (see Pang et al.). All measurements were the average of three technical replicates and error bars show standard deviations.

Soluble PA levels were highly increased in ans-1 and ans-2 mutants while there was nearly 2-fold less insoluble PA present in both ans-1 and ans-2 samples. In contrast, extractable PA content showed only a minor decrease in ldox-1 and ldox-2 mutants. Instead, insoluble PA was highly accumulated with a fold-change increase of ˜1.82 in ldox-1 and ldox-2 samples compared with the wild type (R108) sample. The data suggest that both ANS and LDOX are involved in PA biosynthesis but have distinct functions. However, the seed color of ans or ldox mutants was indistinguishable from wild-type (R108) as shown in FIG. 4C, indicating that PA biosynthesis and oxidation is not disrupted in these single mutants.

3. Analysis of PA Composition in ans and ldox Mutants

To determine whether the composition of PA was altered in mutant seeds, the extracted soluble PAs from the same amount of sample for each genotype were subjected to UPLC/MS (Ultra performance liquid chromatography-mass spectrometry) analyses. UPLC/MS was carried out on an Agilent 1290 Infinity II (Agilent) system equipped with a 6460 triple quadrupole mass spectrometer (Agilent). A XTerra C18 Column, 5 μm, 2.1×250 mm (Waters) was used for separation. The elution procedure was as follows: Solvent A, 0.1% formic acid in water; Solvent B, 0.1% formic acid in methanol; Flow rate, 0.4 mL/min; gradient, 0-1 min, 5% B; 1-2 min, 5% -10% B; 2-13 min, 10%-50% B; 13-14 min, 50%-95% B; 15-15 min, 95% B. The mass spectrometer was set to scan from m/z 100-1000 in negative mode.

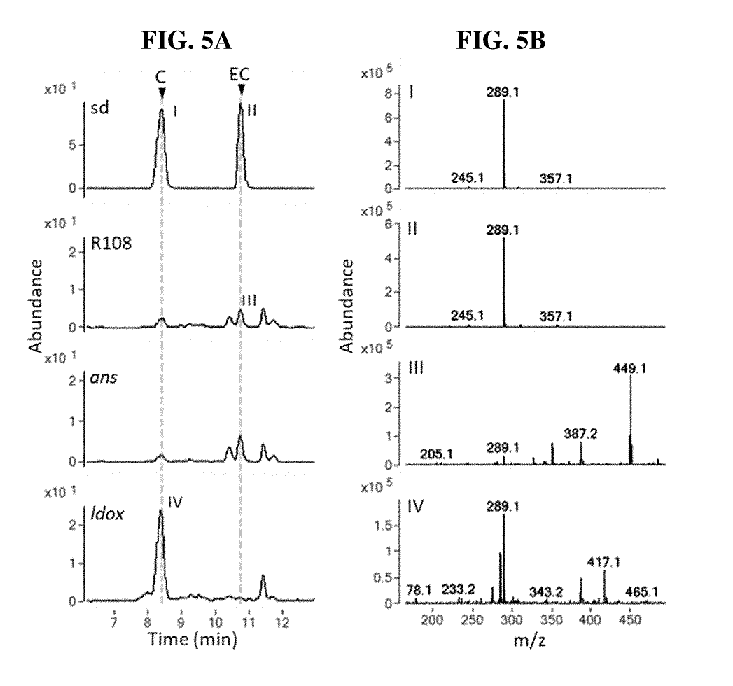

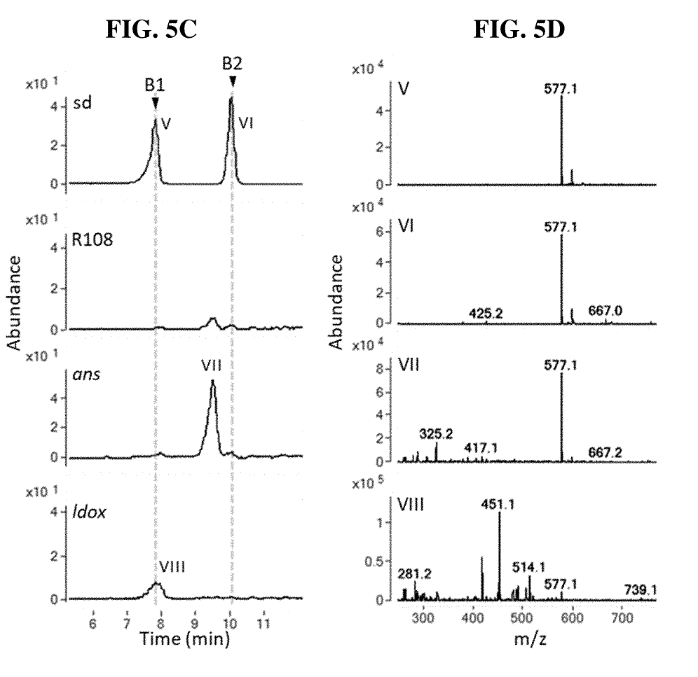

Reactions were analyzed by UPLC/MS in negative mode, and extracted ion chromatograms (EICs) of catechin and epicatechin (m/z 289) for monomer or procyanidin B1 ((+)-catechin-(4β→8)-(−)-epicatechin) and procyanidin B2 ((−)-epicatechin-(4β→8)-(−)-epicatechin) (m/z 577) for dimers, were presented. Results shown in FIGS. 5A and 5B indicate that monomer and dimer composition of soluble PAs was changed in ldox mutant seeds. Peaks I to IV in FIG. 5B were as indicated in FIG. 5A. In wild type (R108) and ans mutant, both epicatechin and lesser amounts of catechin monomers were detected. However, catechin was highly enriched in the ldox mutant whereas the peak corresponding to epicatechin was nearly undetectable in the same samples. FIGS. 5C and 5D shows different profiles for the compounds with the mass corresponding to dimer (type B procyanidin) in ans and ldox mutants. Peaks V-VIII in FIG. 5D were as indicated in FIG. 5C. Soluble PAs were separated by UPLC in FIGS. 5A and 5C with accurate mass detection in FIGS. 5B and 5D. All ions were detected in negative mode. Arrowheads in FIGS. 5A and 5C indicate the peaks for standards. C, catechin; EC, epicatechin; B1, procyanidin B1; B2, procyanidin B2. In wild type (R108), trace amounts of procyanidin B2 were detected. The different retention time of the EIC peak from the standards, procyanidin B1 or procyanidin B2, indicates that the dimer formed in the ans mutant was not composed of only epicatechin or catechin starter and epicatechin extension unit. In the ldox mutant, procyanidin B1 accumulated, suggesting that enriched catechin monomer could be incorporated as starter unit.

To define the monomeric composition and degree of polymerization of soluble PAs, the same amount of extracted PAs from each genotype was subjected to phloroglucinolysis followed by HPLC analysis. Phloroglucinolysis of soluble PA fractions was performed as described by Pang et al. HPLC analysis was carried out on Agilent HP1100 system equipped with diode array detector. A 250 mm×4.6 mm, 5 μm, C18 column was used for separation (Varian Metasil 5 Basic). The elution procedure was as follows: Solvent A (water), Solvent B (methanol), flow rate 1 mL/min. Gradient: 0-5 min, 5% B; 5-20 min, 5% -25% B; 20-40 min, 25%-50% B; 40-50 min, 50%-100% B; 50-60 min, 100% B. Elution profile was monitored at OD 280 nm. To recover the small PA compounds (monomers and dimers) during the purification with Sephadex LH20 resin, the elute from washing with 2 ml 50% MeOH was collected, dried in a speed vacuum centrifuge, dissolved in 50% MeOH, and analyzed by HPLC and UPLC/MS.

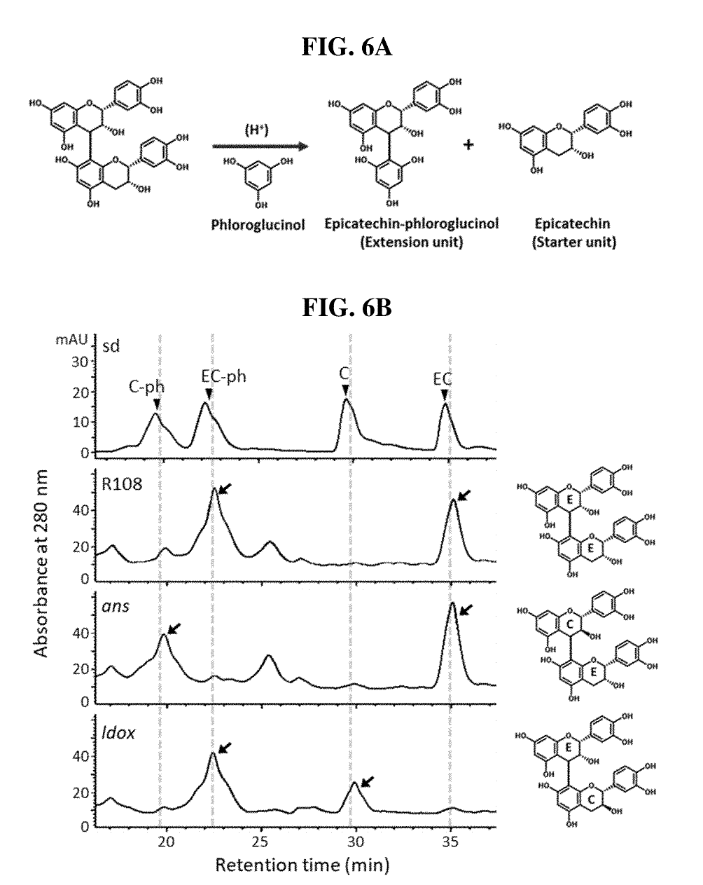

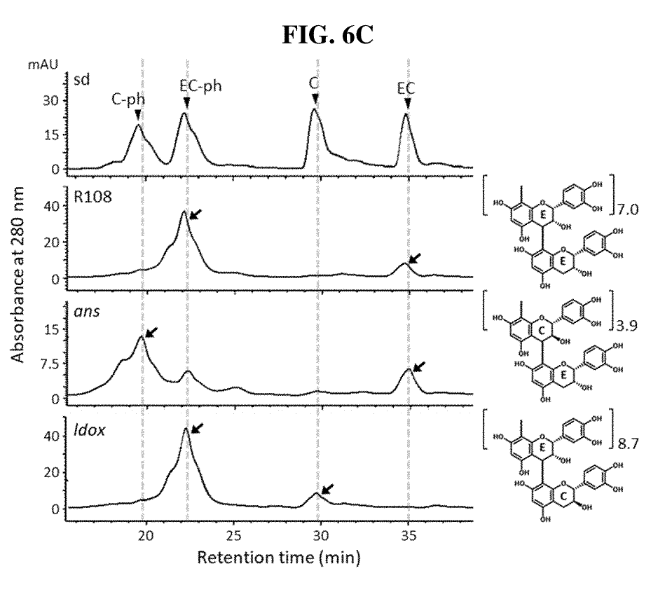

HPLC profiles of phloroglucinolysis products with soluble PAs from wild type (R108) and mutants seeds at 16 DAP are shown in FIGS. 6B and 6C. FIG. 6A shows the mechanism of the phloroglucinol reaction. In FIGS. 6B and 6C, the purified PA fractions after separation on Sephadex LH20 resin from R108 (wild-type), ans and ldox mutant seeds (16 DAP) were hydrolyzed in the presence of phloroglucinol-HCl and used for HPLC analysis. Release of epicatechin-phloroglucinol and epicatechin from procyanidin B2 and catechin-phloroglucinol and catechin from procyanidin B3 in the same assay was analyzed for comparison and is indicated by arrowheads in FIGS. 6B and 6C. Arrows indicate abundant products from phloroglucinolysis for each genotype. Biochemical structure of dimer and polymer for each genotype based on HPLC profile in FIGS. 6B and 6C and mean degree of polymerization (mDP) calculated from the chromatograms in FIG. 6C are presented. C-ph, catechin-phloroglucinol; EC-ph, epicatechin-phloroglucinol; C, catechin; EC, epicatechin; B 1, procyanidin B 1; B2, procyanidin B2. Phloroglucinolysis with the wash fraction of wild type (R108) in FIG. 6B shows that the PAs with small size are mainly composed of epicatechin extension and starter unit. From soluble PAs of the ans mutant, catechin-phloroglucinol conjugate (representing catechin extension units) along with epicatechin was detected. Conversely, samples from the ldox mutant showed peaks corresponding to epicatechin-phloroglucinol conjugate and catechin. Similarly, in HPLC profiles of phloroglucinolysis using the fraction eluted with 50% acetone (for PA oligomers and polymers) (FIG. 6C), more than 90% of epicatechin in extension units appeared to be converted to catechin in the ans mutant, while a change of starter unit from epicatechin to catechin was observed in the ldox mutant. Additionally, more epicatechin-phloroglucinol conjugate was released from the soluble PA fraction of ldox mutants than from wild-type plants, resulting in higher values of mean degree of polymerization (mDP) (wild type, mDP=6.9; ldox, mDP=8.7).Conversely, the peak area of catechin-phloroglucinol combined with the lower amount of epicatechin-phloroglucinol indicated that condensation of PA polymers is less efficient in the ans mutant (mDP=3.9). These data show that loss of function of ANS or LDOX differentially affects the composition of PAs and extent of PA polymerization suggesting that ANS is required to provide epicatechin extension unit and LDOX is required for incorporation of epicatechin at the starter position in the PA conjugation process.

### 4. Biochemical Activity of ANS and LDOX Proteins

The full length ANS and LDOX cDNAs were cloned into pMal-c5x vector (New England Biolabs) at the SalI and BamHI site and BamHI and EcoRI site, respectively. The expression constructs were transformed into E. coli strain NEB® Express Competent E. coli (New England Biolabs). Transformed bacteria were grown in LB medium supplemented with 0.2% glucose to OD 600 of 0.5, and IPTG was added at 0.3 mM to induce protein expression. Bacteria were harvested after 4 h induction. ANS and LDOX proteins were purified with amylose resin (NEB, E8021) following the manufacturer's protocol. Briefly, bacteria were lysed by sonication at 4° C. in extraction buffer (20 mM Tris pH 7.0, 200 mM NaCl, 1 mM DTT). The bacterial lysates were centrifuged at 12,000 g for 15 min at 4° C. The supernatants were loaded on amylose resin which was washed with wash buffer (extraction buffer). Finally, proteins were eluted by elution buffer (20 mM Tris pH 7.0, 200 mM NaCl, 1 mM DTT, 10 mM maltose). Purified proteins were concentrated with an Amicon® Ultra-4 Centrifugal Filter (Millipore) and aliquoted to store at −80° C.

It was previously reported that ANS can convert leucocyanidin to cyanidin. To confirm this, separate enzyme reactions were set up for ANS and LDOX by incubating with leucocyanidin according to Tanner et al. The enzyme reaction was prepared in 100 μL volume including 20 mM potassium phosphate buffer (pH 7.0), 1 mM 2-oxoglutarate, 0.4 mM ammonium iron(II) sulfate, 4 mM sodium ascorbate, 100 mM NaCl, 10 mM maltose, 5 mM DTT and 50 μg recombinant proteins. The reactions were carried out for 1 h at 30° C. and terminated by addition of 1 μL 36% HCl and 10 μL 100% methanol. The reaction mixture was centrifuged for 5 min at 13,000 rpm at 4° C. and analyzed by HPLC. All reverse-phase HPLC analyses were performed on an Agilent HP1100 HPLC using the following gradient: solvent A (1% phosphoric acid) and B (acetonitrile) at 1 ml/min flow rate: 0-5 min, 5% B; 5-10 min, 5-10% B; 10-25 min, 10-17% B; 25-30 min, 17-23% B; 30-65 min, 23-50% B; 65-79 min, 50-100% B; 79-80 min, 100-5% B. Data were collected at 280 and 530 nm for flavonoids and anthocyanidins, respectively. Identifications were based on chromatographic behavior and UV spectra compared with those of authentic standards.

The products of enzyme reactions were further distinguished on an analytical chiral column (catalog no. 80325; Chiral Technologies) on the above HPLC device with the following gradient: solvent A (hexanes with 0.5% acetic acid) and B (ethanol with 0.5% acetic acid) at 1 mL min flow rate: 0 to 20 min, 20% B; 20 to 23 min, 20% to 50% B; 23 to 38 min, 50% B; 38 to 40 min, 50% to 20% B. UV absorption data were collected at 280 nm. Identifications were based on comparison of chromatographic behavior and UV spectra with authentic standards.

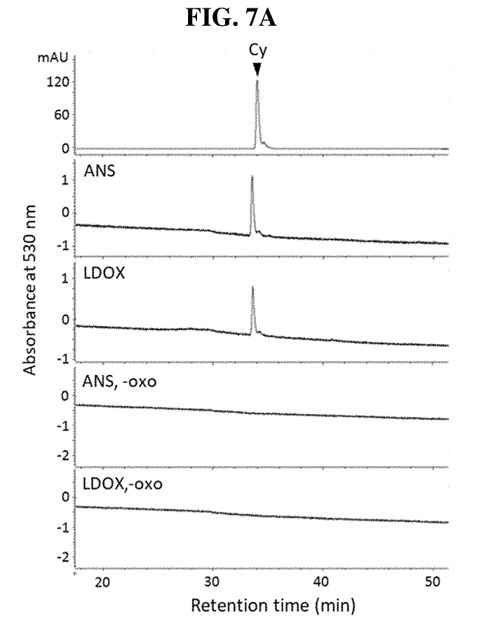

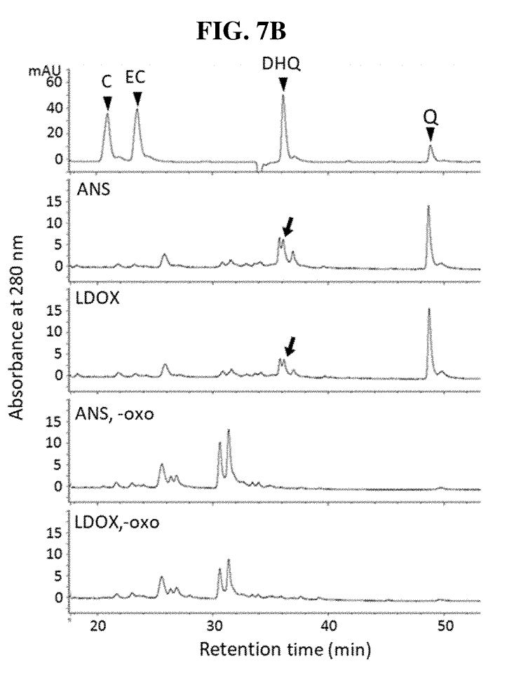

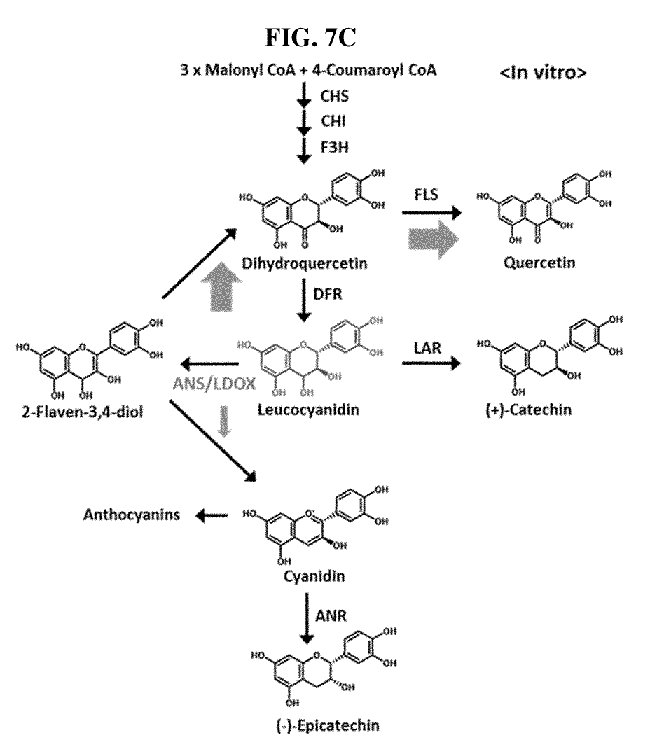

FIG. 7 characterizes an enzyme assay of ANS and LDOX with leucocyanidin. As shown in FIG. 7A, both ANS and LDOX catalyzed in vitro conversion of leucocyanidin to cyanidin. Additionally, a large proportion of the leucocyanidin was converted to dihydroquercetin and quercetin, as seen in FIG. 7B. Enzyme activity appeared to be dependent on 2-oxoglutarate as reported. A 2-flavene-3,4-diol has been suggested as a major nascent product of the reaction of ANS with leucocyanidin, and ANS is also known to catalyze conversion of dihydroquercetin to quercetin in vitro (see Turnbull et al.). In the reactions, LDOX protein showed the same enzymatic activity with leucocyanidin in vitro as ANS. FIG. 7C shows the schematic of ANS or LDOX enzyme reactions with leucocyanidin. The conversion of trans-dihydroquercetin to quercetin is a shared enzyme activity of flavonol synthase (FLS), which suggests that there has been incomplete evolution of substrate selectivity among these proteins or redundancy of enzyme selectivity has been favored evolutionarily. In FIG. 2C, the gray arrows denote the catalyzed reactions in vitro. In FIGS. 7A and 7B, enzyme reaction was performed with leucocyanidin as substrate and recombinant ANS or LDOX protein. The proteins used were noted in each chromatogram. Reactions without 2-oxoglutarate were run as negative controls and denoted as -oxo. Arrowheads in FIGS. 7A and 7B indicate the peaks for standards. Arrows in FIG. 7B indicate the peaks corresponding to dihydroquercetin. Cy, cyanidin; C, catechin; EC, epicatechin; DHQ, dihydroquercetin; Q, quercetin.

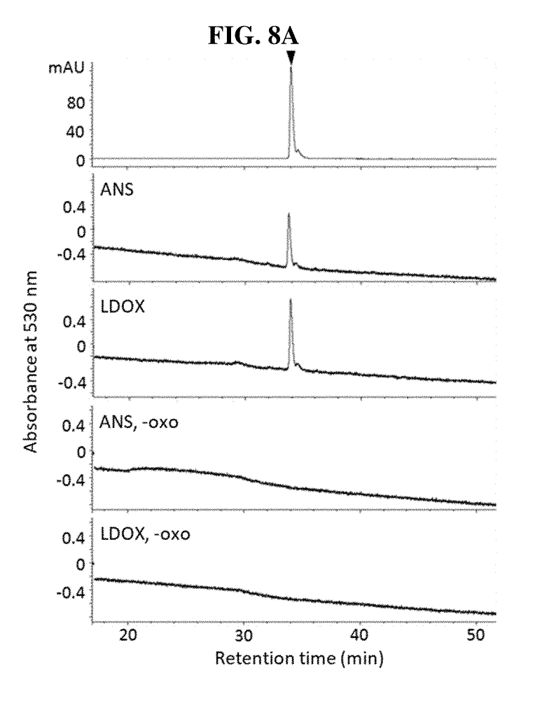

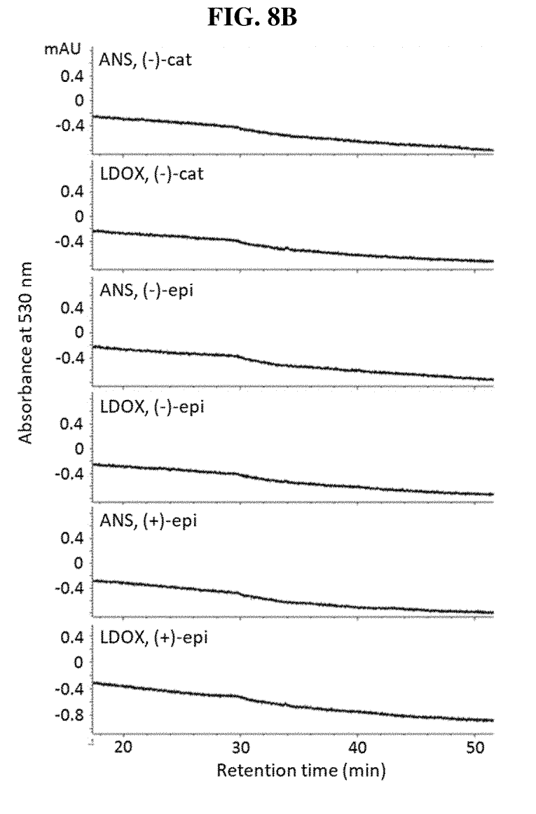

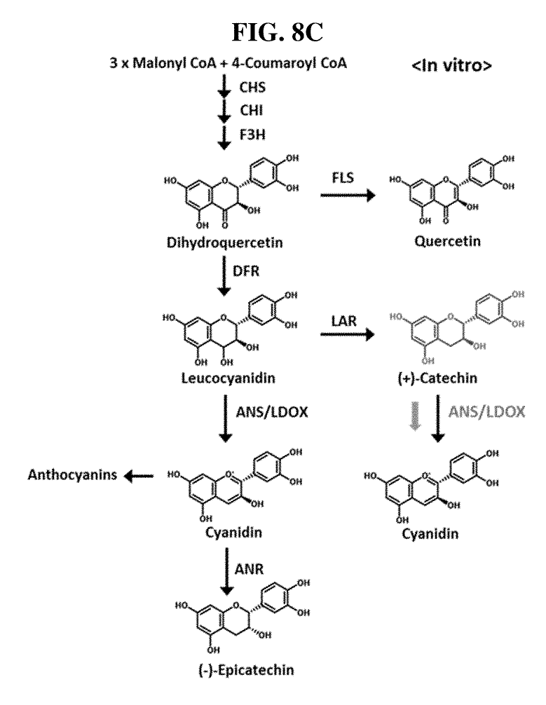

Since catechin was highly accumulated in the ldox mutant, it was tested whether ANS or LDOX protein can react with (+)-catechin (2R, 3S), which is stated as a naturally synthesized monomeric unit of PAs. The same enzyme reaction was set up but using 200 μM of (+)-catechin as substrate instead of leucocyanidin. Reactions without 2-oxoglutarate (-oxo) were run as negative controls. FIG. 8 shows a characterization of the enzyme assay of ANS and LDOX with (+)-catechin. FIG. 8A shows HPLC profiles at absorbance at 530 nm. The enzyme reaction was performed with (+)-catechin as substrate and recombinant ANS or LDOX protein. Arrowhead indicates the peak for cyanidin (Cy) standard. Reactions without 2-oxoglutarate were run as negative controls and are denoted as -oxo. FIG. 8B shows HPLC profiles at absorbance of 530 nm. The enzyme assay was performed with (−)-catechin, (−)-epicatechin or (+)-epicatechin as substrate and purified recombinant ANS or LDOX protein. The substrate in each reaction is indicated. (−)-cat, (−)-catechin; (−)-epi, (−)-epicatechin; (+)-epi, (+)-epicatechin. FIG. 8C shows a scheme of ANS and LDOX reaction with (+)-catechin. The gray arrow denotes the catalyzed reaction in vitro. The proteins used are indicated in each chromatogram.

FIG. 8A shows that both ANS and LDOX catalyze the in vitro conversion of (+)-catechin to cyanidin. Three other flavan-3-ol units, (−)-catechin (2S, 3R), (+)-epicatechin (2S,3S) and (−)-epicatechin (2R,3R), which have same biochemical formula but differ structurally according to the nature of the stereochemistry of the asymmetric carbons on the C ring, were also tested as substrates as shown in FIG. 8B. No cyanidin generation was detected from any of these substrates, indicating that conversion of (+)-catechin to cyanidin by ANS and LDOX is a stereospecific enzymatic reaction as shown in FIG. 8C.

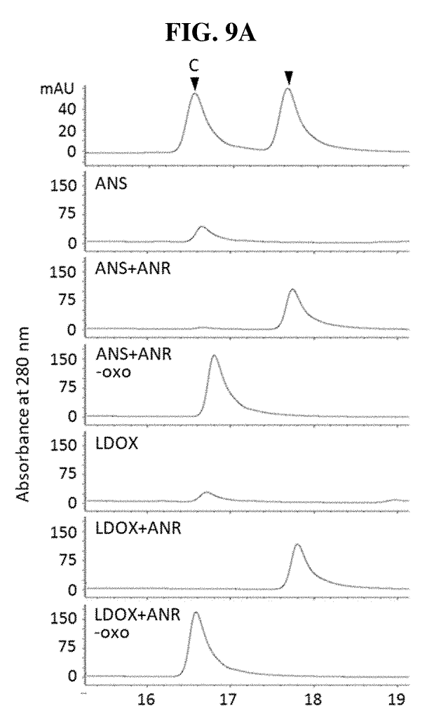

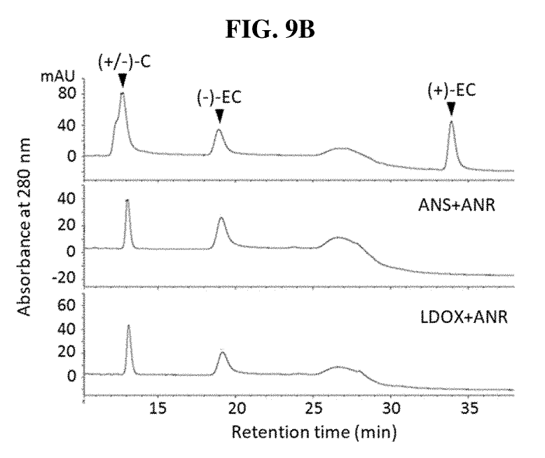

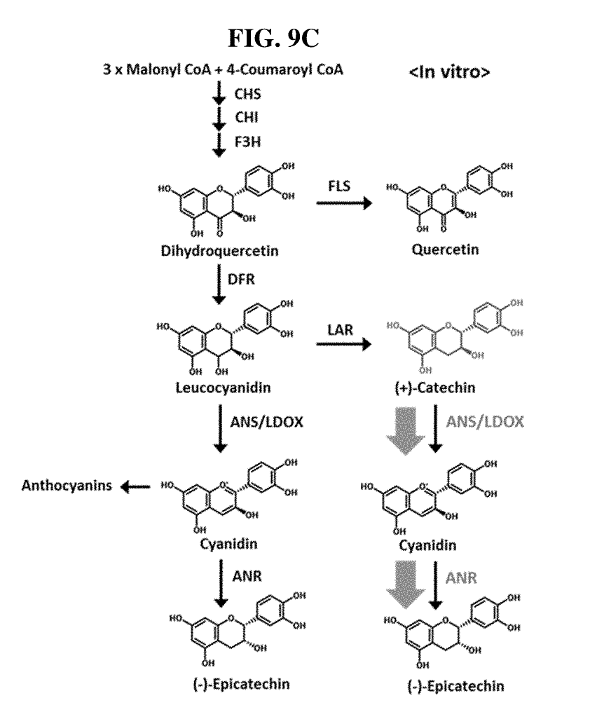

Next, the combined reaction of ANS or LDOX with ANR was tested to check if (+)-catechin can be converted to (−)-epicatechin, which is the almost exclusive monomer unit of PA polymers in Medicago truncatula. The same conditions for enzyme reaction, but additionally including 10 mM NADPH, were used with 200 μM (+)-catechin as substrate and 50 μg purified recombinant ANS and ANR proteins or LDOX and ANR proteins. The same reaction was also performed without 2-ketoglutarate as negative control. In both combination, more than 70% of the (+)-catechin was converted to epicatechin, as shown in FIG. 9A.

FIG. 9 characterizes the enzyme assay of ANS and LDOX combined with ANR protein. FIG. 9A shows HPLC profiles at asbsorbance of 280 nm. The enzyme assay was performed with (+)-catechin as substrate and recombinant ANS or LDOX only or combined with ANR protein. Reactions without 2-oxoglutarate were run as negative controls and denoted as -oxo. FIG. 9B shows HPLC profiles with chiral column for the enzyme assay in FIG. 9A. FIG. 9C shows a scheme of ANS and LDOX reactions combined with ANR with (+)-catechin as substrate. The gray arrow denotes the catalyzed reactions in vitro. The proteins used are indicated in each chromatogram. Arrowheads in FIGS. 9A and 9B indicate the peak for standards. C, catechin; EC, epicatechin; (+/−)-C, (+)-catechin and (−)-catechin; (−)-EC, (−)-epicatechin; (+)-EC, (+)-epicatechin.

To confirm the chirality of the produced epicatechin, the reaction mixture was analyzed by HPLC on a chiral column (Pang 2013), along with authentic standards. FIG. 9B shows that both ANS+ANR and LDOX+ANR combinations produced only (−)-epicatechin, indicating that epicatechin was not formed from enzymatic or non-enzymatic epimerization of (+)-catechin (Xie et al.). A schematic of the conversion of (+)-catechin to (−)-epicatechin by the ANS or LDOX enzyme reactions combined with ANR is shown in FIG. 9C.

5. Genetic Analysis of ldox and lar Mutants

(+)-Catechin is known to be the product of the enzyme reaction catalyzed by LAR with leucocyanidin as substrate. Thus, the combination of ldox and lar mutation was generated to test whether LAR activity is required for (+)-catechin accumulation in the ldox mutant. For genetic crossing (Veerappan et al.), two pairs of fine tip forceps and a straight-edge scalpel were used for keel petal incision, the removal of anthers from the unopened female flower bud and artificial cross-pollination. The petals around the anthers in the pollen donor flower were removed and attached pollens were then gently placed on the tip of the stigma of the female flower multiple times to deposit the pollen grains. The fertilized F1 seeds were harvested and the progenies of F1 plants were analyzed to select the plants with insertion of Tnt1 transposons in both LDOX and LAR genes for further analysis. The Tnt1 insertional mutant of LAR was confirmed by PCR as reported in Liu et al.

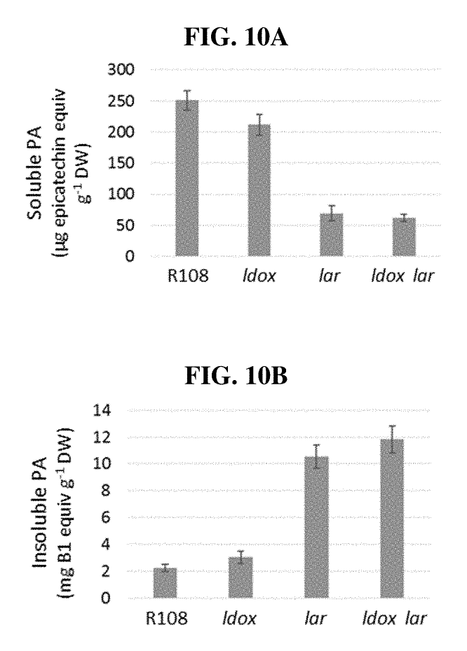

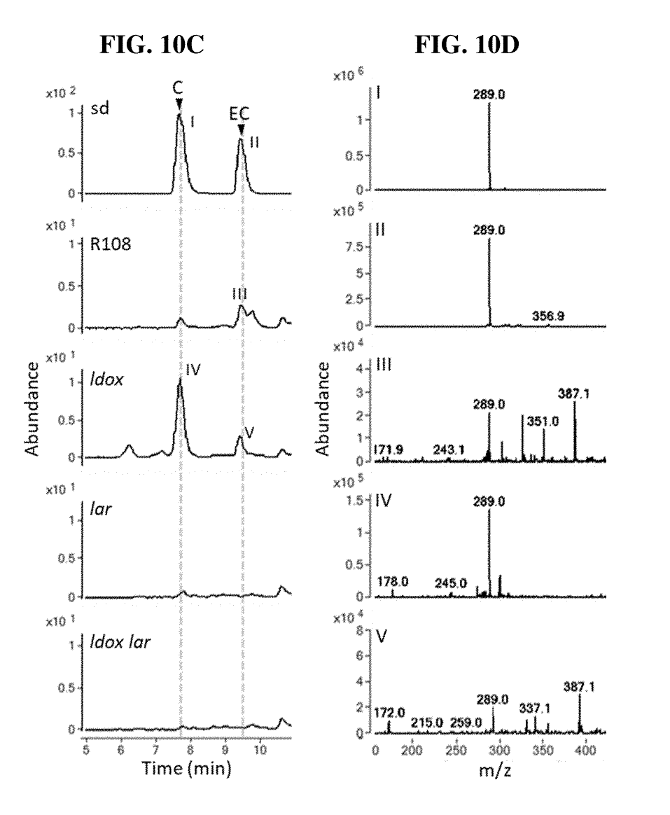

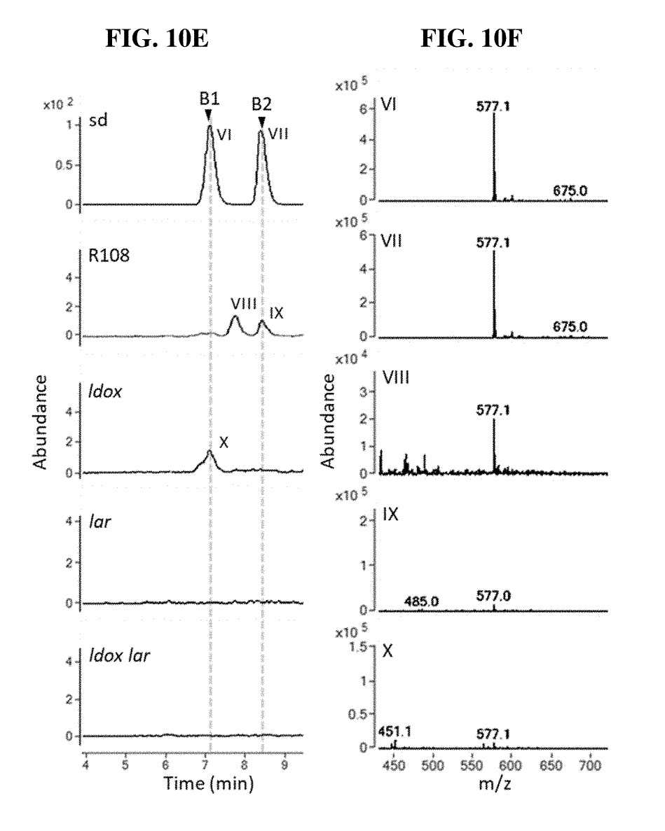

FIG. 10 shows change of PA monomer and dimer composition in ldox lar double mutant. FIG. 10A shows soluble PA levels in wild type (R108), ldox, lar and ldox lar 4-DAP pods as quantified with DMACA reagent and expressed as epicatechin equivalents. Error bars represent SD of three technical replicates. FIG. 10B shows insoluble PA levels in wild type (R108), ldox, lar and ldox lar 4-DAP pods as quantified by the butanol-HCl method and expressed as procyanidin B1 equivalents. Error bars represent SD of three technical replicates. FIG. 10C shows EIC of epicatechin and catechin monomers (m/z 289) in soluble PAs extracted from wild type (R108), ldox, lar and ldox lar 4-DAP pods. FIG. 10D shows mass spectra of peaks in FIG. 10C. Peaks I to V are as indicated in FIG. 10C. FIG. 10E shows EIC of procyanidin dimers (m/z 577) in soluble PAs extracted from R108, ldox, lar and ldox lar 4-DAP pods. FIG. 10F shows mass spectra of peaks in FIG. 10E. Peaks VI to X are as indicated in FIG. 10E. Soluble PAs were separated by UPLC in FIGS. 10C and 10E with accurate mass detection in FIGS. 10D and 10F. All ions were detected in negative mode. Arrowheads in FIGS. 10C and 10E indicate the peaks for standards. C, catechin; EC, epicatechin; B 1, procyanidin B 1; B2, procyanidin B2

FIG. 10A shows soluble PAs quantified by the DMACA method with their contents expressed as epicatechin equivalents. FIG. 10B shows insoluble PAs quantified by the butanol/HCl method with their contents expressed as procyanidin B2 equivalents. At 4 DAP, loss of function of LAR gave a large reduction in soluble PAs but large increase of insoluble PAs compared to wild type, as reported in Liu et al. In the ldox lar double mutant, similar level of soluble and insoluble PA accumulation was detected as in the lar mutant (FIGS. 10A and 10B). LC/MS analysis with soluble extracts showed that the ldox lar double mutant produced PAs with similar monomer and dimer composition as the lar single mutant which has epicatechin monomer and dimer but almost no catechin, in contrast to the ldox mutant with high accumulation of catechin and procyanidin B1 dimer composed of catechin starter and epicatechin extension unit. The data indicate that LDOX is functioning downstream of LAR protein on the same pathway and LAR activity is required to show the ldox mutant phenotype.

5. Catechin-Based PA Generation in the ans and ldox Double Mutant

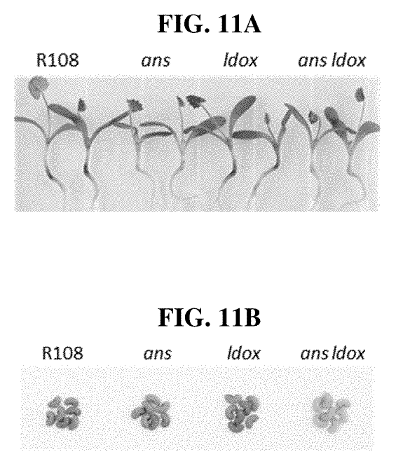

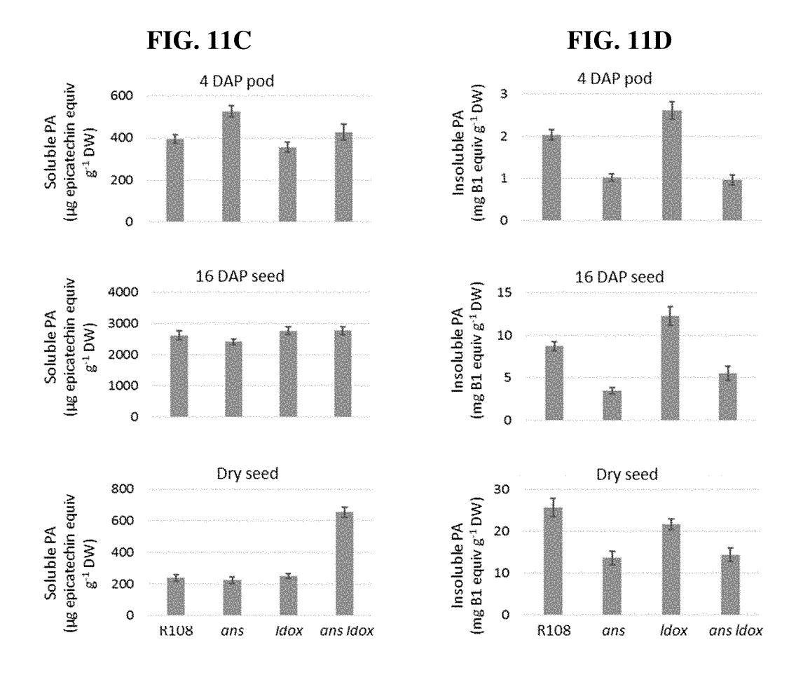

Since loss of function mutations in ANS and LODX differently affected PA extension and starter units, the double knock-out mutant of ans and ldox was generated to check how PA composition would be changed. FIG. 11 characterizes the phenotype of ans ldox double mutants. FIG. 11A shows anthocyanin accumulation in wild type (R108), ans, ldox and ans ldox 7-days old seedlings. FIG. 11B shows seed coat phenotypes of wild type (R108), ans, ldox and ans ldox dry seeds. FIG. 11C shows soluble PA levels in wild type (R108), ans, ldox, and ans ldox 4-DAP pods, 16-DAP and dry seeds as quantified with DMACA reagent and expressed as epicatechin equivalents. Error bars represent SD of three technical replicates. FIG. 11D shows soluble PA levels in wild type (R108), ans, ldox, and ans ldox 4-DAP pods, 16-DAP and dry seeds quantified by the butanol-HCl method and expressed as procyanidin B1 equivalents. Error bars represent SD of three technical replicates.

As shown in FIG. 11A, seedlings of the ans ldox double mutant had no anthocyanin accumulation, similar to ans mutant plants. In contrast, wild type and ldox mutant seedlings accumulated anthocyanin in the hypocotyl and junction of hypocotyl and root at 7 days after germination on B5 medium, indicating that anthocyanin accumulation in the seedling stage is dependent on ANS gene expression. Comparison of seeds after desiccation in FIG. 11B showed that the ans ldox double mutant seeds have a brighter color compared to other genotypes, indicating that PA and/or anthocyanin content is reduced in the seed coat or changes of PA composition in the double mutant affect the progress or extent of PA oxidation.

In FIGS. 11C and 11D, the accumulation of soluble PAs and insoluble PAs were measured with entire young pods (4 DAP), developing seeds (16DAP) and desiccated seeds of R108 (wild type), ans, ldox and ans ldox mutants. Soluble PA contents were measured by the DMACA method and expressed as epicatechin equivalents. Insoluble PA contents were measured by the butanol/HCl method and expressed as procyanidin B2 equivalents (Pang et al.). All measurements were the average of three technical replicates and error bars show standard deviations. Soluble PA level was significantly increased in ans and ans ldox double mutants at the very early stage of seed development (4 DAP). Soluble PA level was most enhanced in dry seeds of the ans ldox double mutant, which showed nearly 3-fold increase compared to other genotypes. Insoluble PA level was consistently decreased in both ans and ans ldox seeds in every developmental stage. In the ldox mutant, more insoluble PA was accumulated at the young stage (˜16 DAP) with a fold-change increase of ˜1.82 in mutant compared with the wild type (R108). Even though the extent of increase was less than that in lar mutant seeds, the data support the idea that LDOX function is related to LAR activity on the PA biosynthetic pathway.

PA quantity in the ans ldox double mutant quantity was similar to that in the ans mutant, especially for insoluble PA content throughout seed maturation and soluble PA content at the early developmental stage. This suggests that LDOX is hyponastic to ANS for the accumulation of insoluble PA, meaning that ANS activity is required to efficiently provide extension units of (insoluble) PA polymers. The data are also consistent with the change of extension unit from epicatechin to catechin, as observed in both ans and ans ldox mutants.

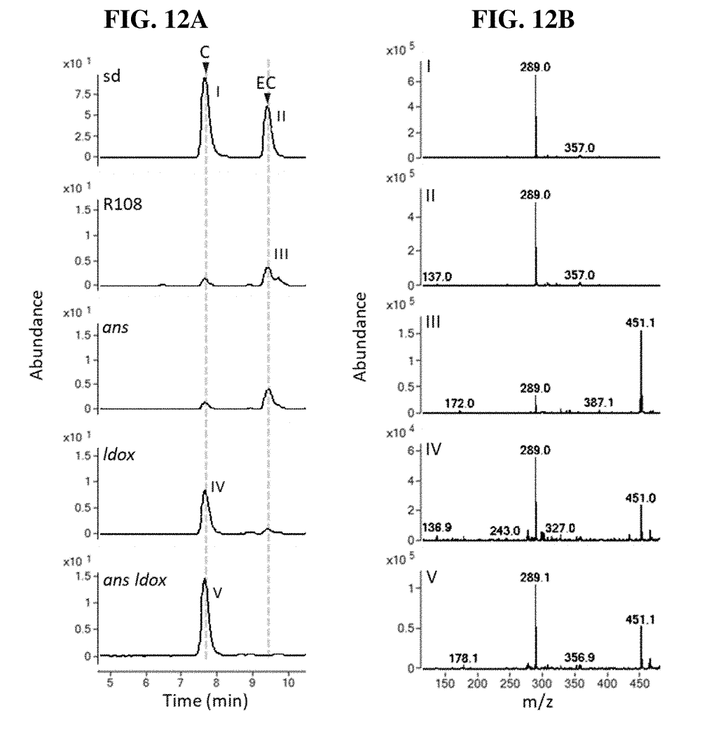

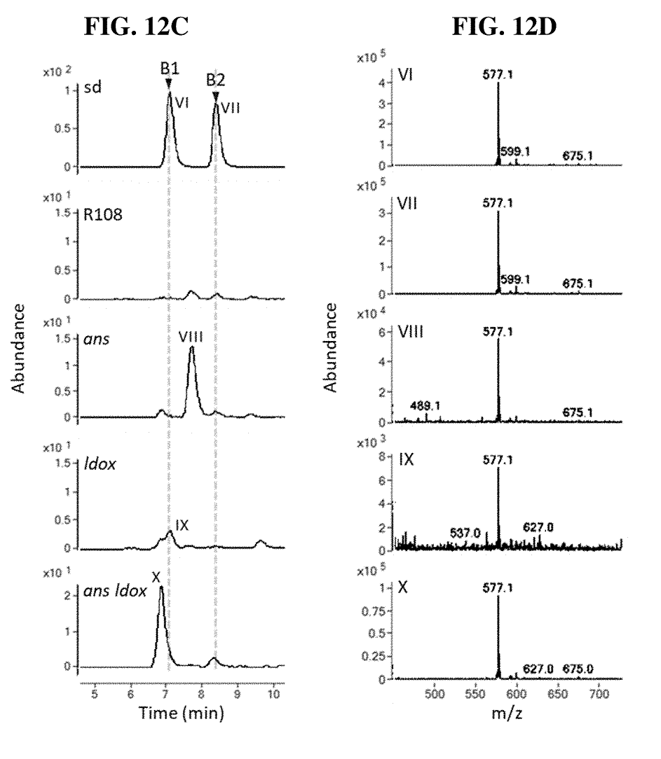

To determine whether the composition of PAs was altered in mutant seeds, the extracted soluble PAs from same amount of samples were subjected to UPLC/MS analyses. Reactions were analyzed by UPLC/MS in negative mode, and extracted ion chromatograms (EICs) of catechin (C) and epicatechin (EC), (m/z 289) for monomer or procyanidin B1 and procyanidin B2 equivalent (m/z 577) for dimers, are presented. FIG. 12 characterizes change of PA monomer and dimer composition in ans ldox double mutant. FIG. 12A shows EIC of epicatechin and catechin monomers (m/z 289) in soluble PAs extracted from R108, ans, ldox and ans ldox 4-DAP pods. FIG. 12B shows mass spectra of peaks in FIG. 12A. Peaks I to V are as indicated in FIG. 12A. FIG. 12C shows EIC of procyanidin dimers (m/z 577) in soluble PAs extracted from R108, ans, ldox and ans ldox 4-DAP pods. FIG. 12D shows mass spectra of peaks in FIG. 12C. Peaks VI˜X are as indicated in FIG. 12C. Soluble PAs were separated by UPLC in FIGS. 12A and 12C with accurate mass detection in FIGS. 12B and 12D. All ions were detected in negative mode. Arrowheads in FIGS. 12A and 12C indicate the peaks for standards. C, catechin; EC, epicatechin; B 1, procyanidin B 1; B2, procyanidin B2.

Results shown in FIGS. 12A and 12B indicate that monomer composition of soluble PAs in the ans ldox double mutant is similar to that in the ldox mutant. In wild type (R108), both catechin and epicatechin monomer were detected, which was similar to the situation in the ans mutant, but catechin monomer was highly enriched along with reduction of epicatechin in the ldox mutant. In the ans ldox double mutant, catechin peak area was more increased than in the ldox single mutant, accompanied by nearly complete loss of epicatechin generation.

FIGS. 12C and 12D show that compounds with mass corresponding to procyanidin dimer (type B procyanidin) were highly accumulated in the ans ldox double mutant as well as in the ans single mutant. The different retention time of the EIC peak from those of the standards procyanidin B 1, procyanidin B2 and procyanidin B4 (which is the dimer accumulated in the ans mutant based on phloroglucinolysis assay in FIG. 6) indicates that the dimer formed in the ans ldox double mutant is possibly procyanidin B3, composed of only catechin subunits.

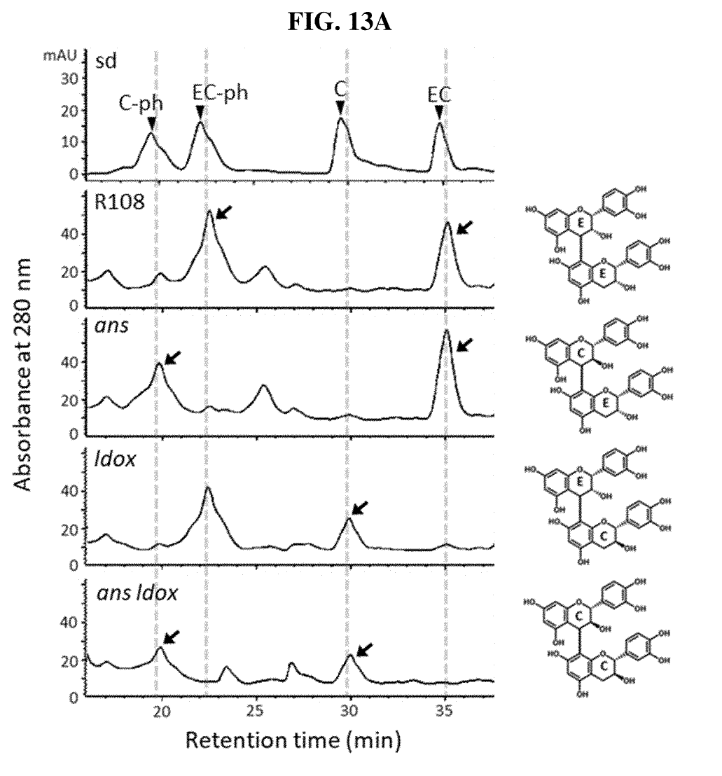

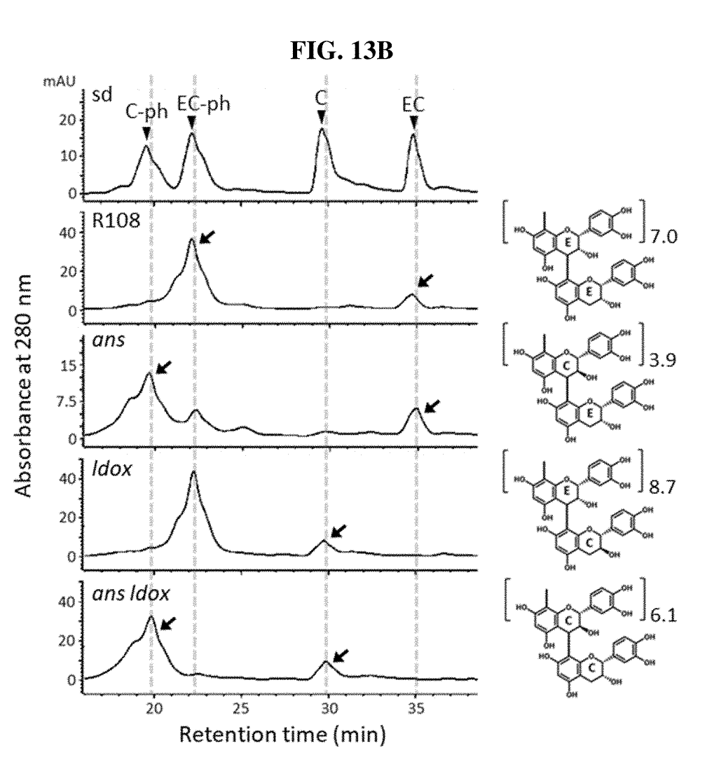

To define the monomeric composition and degree of polymerization of soluble PAs, the same amount of extracted PAs were subjected to phloroglucinolysis followed by HPLC analysis. Release of epicatechin-phloroglucinol and epicatechin from procyanidin B2 and catechin-phloroglucinol and catechin from procyanidin B3 was analyzed for comparison. HPLC profiles of phloroglucinolysis products with soluble PAs from wild type (R108) and mutant seeds at 16 DAP are shown in FIG. 13. FIG. 13 characterizes change of PA composition in ans and ldox mutants. FIG. 13A shows phloroglucinolysis of soluble PAs (50% methanol-washing fraction) from R108 (wild-type), ans, ldox and ans ldox seeds. FIG. 13B shows phloroglucinolysis of soluble PAs (50% acetone-elution fraction) from R108 (wild-type), ans, ldox and ans ldox seeds. In FIGS. 13A and 13B, purified PAs fractionated on Sephadex LH20 resin from R108 (wild-type), ans and ldox mutant seeds (16 DAP) were hydrolyzed in the presence of phloroglucinol-HCl and used for HPLC analysis. Release of epicatechin-phloroglucinol and epicatechin from procyanidin B2 and catechin-phloroglucinol and catechin from procyanidin B3 in the same assay was analyzed for comparison and indicated by arrowheads in FIGS. 13A and 13B. Arrows indicate abundant products from phloroglucinolysis for each genotype. Biochemical structure of dimer and polymer for each genotype based on HPLC profile in FIGS. 13A and 13B and mean degree of polymerization (mDP) calculated from the chromatogram in FIG. 13B are presented. C-ph, catechin-phloroglucinol; EC-ph, epicatechin-phloroglucinol; C, catechin; EC, epicatechin; B 1, procyanidin B 1; B2, procyanidin B2.

Phloroglucinolysis of the wash fraction of the double mutant in FIG. 13A confirmed that PA monomer and dimer from the double mutant were almost exclusively composed of catechin subunit. Similarly, in HPLC profiles of phloroglucinolysis products using the elute fraction with 50% acetone (for PA oligomers or polymers), both extension unit and starter unit appeared to be changed from epicatechin to catechin in the ans ldox double mutant. The data indicate that loss of function of both ANS and LDOX changes the composition of Medicago PAs from epicatechin-based compounds to catechin-based flavan-3-ol polymers. The additive effect of the ans and ldox mutations suggests that the composition of PAs is dependent on separate activities of ANS and LDOX proteins.

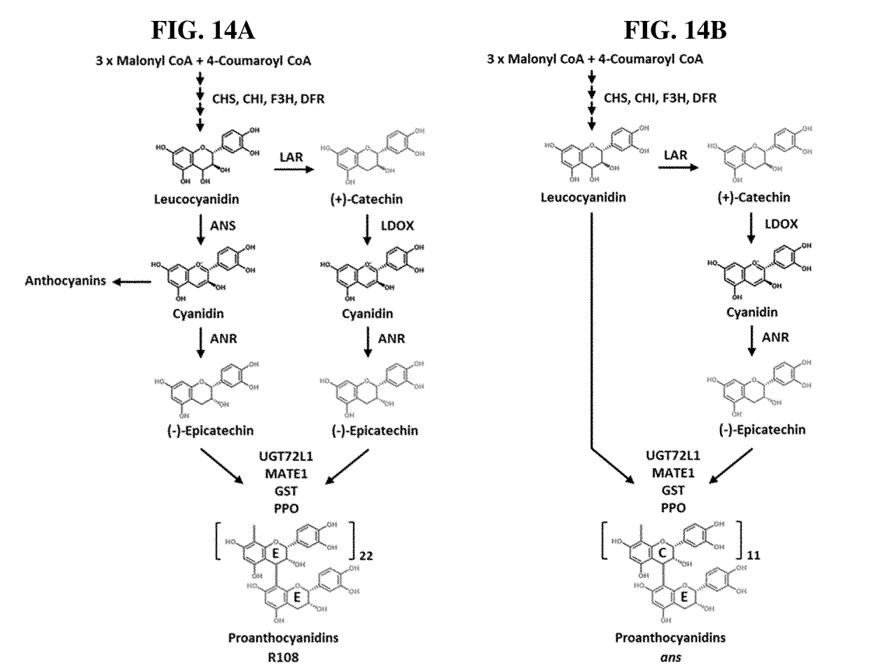

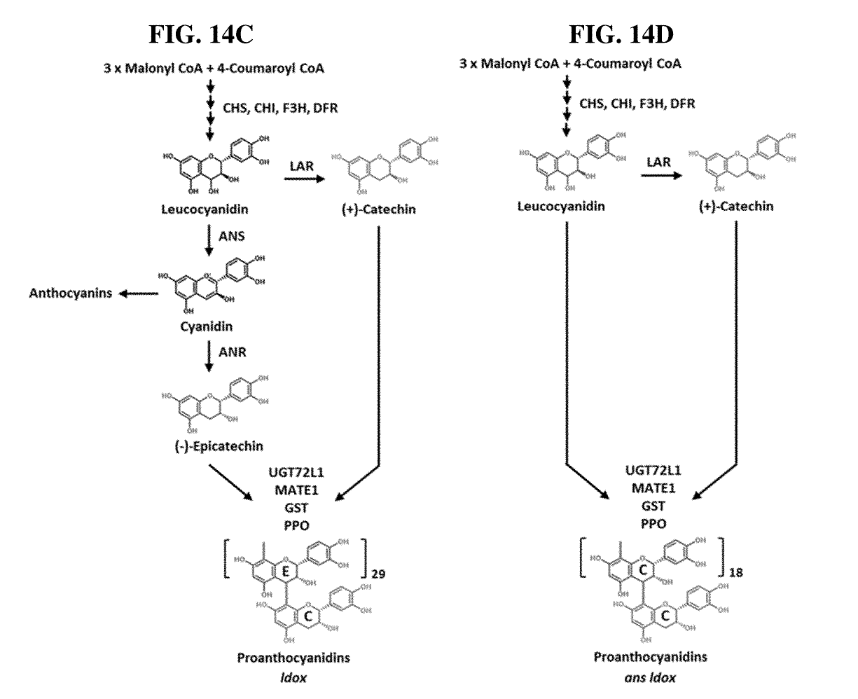

FIG. 14 shows the proposed model of ANS and LDOX function during PA biosynthesis. FIG. 14. A summary of PA biosynthesis is shown in FIG. 14A for wild type (R108), and in FIG. 14B for ans, FIG. 14C for ldox and FIG. 14D for ans ldox double mutants. The separate contribution of ANS and LDOX proteins in the parallel pathways to provide starter and extension unit of PAs is proposed based on genetic and biochemical analysis of wild type (R108), ans, ldox, and ans ldox plants. The change of PA composition in each genotype is summarized and mDP of dry seeds is indicated. It is possible that leucocyanidin and (+)-catechin are incorporated as extension unit and starter unit in the absence of ANS and LDOX activity, respectively. The model explains how PAs are exclusively composed of epicatechin subunits in Medicago truncatula in spite of the expression of LAR

The change of PA composition in each genotype is described based on genetic and biochemical analysis. The separate contribution of ANS and LDOX proteins in the parallel pathways to provide starter and extension units explain how PAs are exclusively composed of epicatechin subunits in Medicago truncatula. Also, the changes of soluble and insoluble PA contents and seed color in the mutants indicate that the composition might be one of the important factors to decide PA quality such as solubility and oxidation state of PAs.

### 6. Relevant Sequences

Mt ANS (Medtr5g011250.1) cDNA
(SEQ ID NO: 11)
ATGGGAACGGTGGCTCAAAGAGTTGAAAGCTTAGCCTTGAGTGGTATATC
ATCAATCCCAAAAGAATATGTGAGACCAAAAGAAGAGTTAGCAAACATAG
GTAACATCTTTGATGAAGAAAAAAAAGAAGGTCCTCAAGTTCCAACAATA
GACCTAAAAGAAATAAACTCTTCAGATGAAATAGTTAGAGGAAAATGTAG
AGAGAAGCTTAAGAAAGCTGCAGAAGAATGGGGTGTGATGCATTTAGTGA
ACCATGGTATATCTGATGATCTTATTAATCGTTTGAAGAAAGCTGGTGAA
ACATTTTTTGAGCTTCCTGTTGAAGAAAAAGAGAAATATGCAAATGATCA
AAGTTCTGGGAAGATTCAAGGTTATGGAAGTAAATTAGCTAATAATGCTA
GTGGTCAACTTGAATGGGAAGATTATTTCTTTCATTGCATTTTTCCAGAG
GATAAACGTGATTTGTCTATATGGCCCAAGACACCTGCTGATTATACTAA
GGTTACAAGCGAATATGCAAAGGAATTAAGAGTCCTAGCTAGCAAGATAA
TGGAAGTGTTATCTCTTGAACTAGGGTTGGAAGGTGGAAGGTTAGAAAAA
GAAGCTGGTGGAATGGAAGAGCTTTTACTTCAAATGAAAATTAATTACTA
CCCAATTTGCCCTCAACCAGAGCTAGCACTTGGAGTTGAAGCCCATACAG
ATGTAAGTTCACTTACTTTCCTCCTCCATAATATGGTGCCAGGTTTGCAA
CTTTTCTATGAGGGCAAATGGGTCACTGCAAAATGTGTACCTGATTCAAT
TCTCATGCATATTGGTGACACAATTGAGATACTTAGCAATGGAAAGTACA
AAAGTATCCTTCATCGTGGATTGGTGAATAAGGAAAAAGTTAGAATATCT
TGGGCAGTGTTTTGTGAACCACCTAAGGAGAAAATTATTCTGAAGCCACT
TCCTGAACTTGTAACTGAAAAAGAACCAGCAAGGTTTCCACCTCGCACTT
TTGCTCAGCATATTCATCACAAACTTTTTAGGAAGGATGAGGAGGAGAAG
AAAGATGATCCCAAAAAATGA
Mt ANS (Medtr5g011250.1) protein
(SEQ ID NO: 12)
MGTVAQRVESLALSGISSIPKEYVRPKEELANIGNIFDEEKKEGPQVPTI
DLKEINSSDEIVRGKCREKLKKAAEEWGVMHLVNHGISDDLINRLKKAGE
TFFELPVEEKEKYANDQSSGKIQGYGSKLANNASGQLEWEDYFFHCIFPE
DKRDLSIWPKTPADYTKVTSEYAKELRVLASKIMEVLSLELGLEGGRLEK
EAGGMEELLLQMKINYYPICPQPELALGVEAHTDVSSLTFLLHNMVPGLQ
LFYEGKWVTAKCVPDSILMHIGDTIEILSNGKYKSILHRGLVNKEKVRIS
WAVFCEPPKEKIILKPLPELVTEKEPARFPPRTFAQHIHHKLFRKDEEEK
KDDPKK
Mt LDOX (Medtr3g072810.1) cDNA
(SEQ ID NO: 13)
ATGGAAGTCAAAAGAGTACAAACTTTAGCTTGTAATCAGCTAAAGGAGCT
TCCACCACAATTCATTCGCTTAGCAAATGAAAGGCCAGAGAATACAAAGG
CCATGGAGGGTGTTACTGTGCCTATGATTTCATTGTCTCAACCACATAAC
CTTTTAGTGAAGAAAATCAATGAAGCTGCTTCTGAGTGGGGTTTCTTTGT
GATCACTGACCATGGTATATCTCAAAAACTTATTCAAAGTTTGCAAGATG
TGGGCCAGGAGTTTTTTTCTCTCCCTCAAAAGGAGAAAGAGACATATGCA
AATGATCCATCTAGTGGTAAATTTGATGGCTATGGAACAAAGATGACCAA
GAACCTTGAACAAAAGGTTGAGTGGGTTGATTATTATTTTCATCTCATGT
CTCCTCATTCTAAGTTGAATTTTGAGATGTGGCCCAAAAGTCCTCCTTCG
TACAGGGAAGTAGTACAAGAATACAATAAAGAGATGTTAAGGGTGACAGA
CAATATTTTGGAGCTTTTGTCTGAAGGACTAGAATTGGAGAGTAAGACTT
TGAAGTCTTGTTTGGGAGGTGAAGAAATAGAATTAGAAATGAAGATAAAT
ATGTATCCACCATGTCCACAACCTGAATTGGCATTAGGAGTTGAGCCCCA
TACTGATATGTCTGCCATTACACTACTTGTTCCAAATGATGTTCCAGGCC
TTCAAGTTTGGAAGGACAATAATTGGGTTGCAGTAAATTACTTGCAAAAT
GCACTCTTTGTGCACATTGGTGACCAACTTGAGGTGTTGAGCAATGGGAG
GTACAAGAGTGTCTTGCACAGAAGCTTGGTGAACAAGGAACGCAAGCGTA
TGTCCTGGGCAGTATTTGTTGCTCCTCCACATGAGGTCGTGGTTGGACCT
CTTCCTCCGCTCGTCAATGATCAAAACCCTGCCAAATTTTCAACAAAAAC
CTATGCTGAGTATCGCTATCGCAAATTCAACAAGATTCCCCAGTAG
Mt LDOX (Medtr3g072810.1) protein
(SEQ ID NO: 14)
MEVKRVQTLACNQLKELPPQFIRLANERPENTKAMEGVTVPMISLSQPHN
LLVKKINEAASEWGFFVITDHGISQKLIQSLQDVGQEFFSLPQKEKETYA
NDPSSGKFDGYGTKMTKNLEQKVEWVDYYFHLMSPHSKLNFEMWPKSPPS
YREVVQEYNKEMLRVTDNILELLSEGLELESKTLKSCLGGEEIELEMKIN
MYPPCPQPELALGVEPHTDMSAITLLVPNDVPGLQVWKDNNWVAVNYLQN
ALFVHIGDQLEVLSNGRYKSVLHRSLVNKERKRMSWAVFVAPPHEVVVGP
LPPLVNDQNPAKFSTKTYAEYRYRKFNKIPQ

### REFERENCES CITED

The following documents and publications are hereby incorporated by reference.

### Non-Patent Publications

Gonzalez-Centeno M R, Jourdes M, Femenia A, Simal S, Rossello C, Teissedre P L: Proanthocyanidin composition and antioxidant potential of the stem winemaking byproducts from 10 different grape varieties (Vitis vinifera L.). J Agric Food Chem 2012, 60:11850-11858.
Huang Y F, Doligez A, Fournier-Level A, Le Cunff L, Bertrand Y, Canaguier A, Morel C, Miralles V, Veran F, Souquet J M, et al.: Dissecting genetic architecture of grape proanthocyanidin composition through quantitative trait locus mapping. BMC Plant Biol 2012, 12:30.
Jorgensen E M, Marin A B, Kennedy J A: Analysis of the oxidative degradation of proanthocyanidins under basic conditions. Journal of Agricultural and Food Chemistry 2004, 52:2292-2296.
Tadege M, Wen J, He J, Tu H, Kwak Y, Eschstruth A, Cayrel A, Endre G, Zhao P X, Chabaud M, et al.: Large-scale insertional mutagenesis using the Tnt1 retrotransposon in the model legume Medicago truncatula. Plant J 2008, 54:335-347.
Pang Y Z, Peel G J, Sharma S B, Tang Y H, Dixon R A: A transcript profiling approach reveals an epicatechin-specific glucosyltransferase expressed in the seed coat of Medicago truncatula. Proceedings of the National Academy of Sciences of the United States of America 2008, 105:14210-14215.
Tanner G J, Kristiansen K N: Synthesis of 3,4-cis-[3H]leucocyanidin and enzymatic reduction to catechin. Anal Biochem 1993, 209:274-277.
Turnbull J J, Sobey W J, Aplin R T, Hassan A, Firmin J L, Schofield C J, Prescott A G: Are anthocyanidins the immediate products of anthocyanidin synthase? Chemical Communications 2000:2473-2474.
Pang Y, Abeysinghe I S, He J, He X, Huhman D, Mewan K M, Sumner L W, Yun J, Dixon R A: Functional characterization of proanthocyanidin pathway enzymes from tea and their application for metabolic engineering. Plant Physiol 2013, 161:1103-1116.
Xie D Y, Sharma S B, Paiva N L, Ferreira D, Dixon R A: Role of anthocyanidin reductase, encoded by BANYULS in plant flavonoid biosynthesis. Science 2003, 299:396-399.
Veerappan V, Kadel K, Alexis N, Scott A, Kryvoruchko I, Sinharoy S, Taylor M, Udvardi M, Dickstein R: Keel petal incision: a simple and efficient method for genetic crossing in Medicago truncatula. Plant Methods 2014, 10:11.
Liu C, Wang X, Shulaev V, Dixon R A: A role for leucoanthocyanidin reductase in the extension of proanthocyanidins. Nat Plants 2016, 2:16182.

What is claimed is:

1. A method for producing a modified plant having modified proanthocyanidin (PA) content in cells of the plant compared to an unmodified plant of the same species, comprising:
reducing or eliminating expression of the leucoanthocyanidin dioxygenase (ldox) gene in plant cells; and
using the plant cells to produce a modified plant having reduced or eliminated expression of the leucoanthocyanidin dioxygenase (ldox) gene and modified proanthocyanidins (PAs) in cells of the modified plant, wherein the modified proanthocyanidins (PAs) comprise starter units consisting of catechin and extension units consisting of epicatechin.

2. The method of claim 1, wherein the step of reducing or eliminating expression of the leucoanthocyanidin dioxygenase (ldox) gene comprises introducing a mutation into a leucoanthocyanidin dioxygenase (ldox) gene in substantially all cells of the modified plant, wherein the mutation results in reduced or eliminated expression of the leucoanthocyanidin dioxygenase (ldox) gene in the modified plant.

3. The method of claim 1, wherein the plant is a Medicago truncatula plant.

4. The method of claim 1, wherein the plant is alfalfa, clover, soybean, grape, cacao, tea or strawberry.

5. The method of claim 1, wherein the modified plant has increased insoluble proanthocyanidin (PA) content compared to unmodified plants of the same species.

6. The method of claim 1, wherein the modified plant has reduced astringency compared to unmodified plants of the same species.

7. A modified plant having modified proanthocyanidins (PAs), wherein the modified proanthocyanidins (PAs) comprise starter units consisting of catechin and extension units consisting of epicatechin, wherein substantially all cells of the modified plant comprise a mutation in a leucoanthocyanidin dioxygenase (ldox) gene found in the cells of the modified plant, and wherein the mutation results in reduced or eliminated expression of the leucoanthocyanidin dioxygenase (ldox) gene.

8. A seed of the modified plant of claim 7.

9. The modified plant of claim 7, wherein the modified plant is a Medicago truncatula plant.

10. The modified plant of claim 7, wherein the plant is alfalfa, clover, soybean, grape, cacao, tea or strawberry plant.

11. A method for producing a modified plant having modified proanthocyanidin (PA) content in cells of the plant compared to an unmodified plant of the same species, comprising:
reducing or eliminating expression of the anthocyanidin synthase (ans) gene in plant cells; and
using the plant cells to produce a modified plant having reduced or eliminated expression of the anthocyanidin synthase (ans) gene and modified proanthocyanidins (PAs) in cells of the modified plant, wherein the modified proanthocyanidins (PAs) comprise starter units consisting of epicatechin and extension units consisting of catechin.

12. The method of claim 11, wherein the step of reducing or eliminating expression of the anthocyanidin synthase (ans) gene comprises introducing a mutation into a anthocyanidin synthase (ans) gene in substantially all cells of the modified plant, wherein the mutation results in reduced or eliminated expression of the anthocyanidin synthase (ans) gene in the modified plant.

13. The method of claim 11, wherein the plant is a Medicago truncatula plant.

14. The method of claim 11, wherein the plant is alfalfa, clover, soybean, grape, cacao, tea or strawberry.

15. The method of claim 11, wherein the modified plant has increased soluble proanthocyanidin (PA) content compared to unmodified plants of the same species.

16. A modified plant having modified proanthocyanidins (PAs), wherein the modified proanthocyanidins (PAs) comprise starter units consisting of epicatechin and extension units consisting of catechin, wherein substantially all cells of the modified plant comprise a mutation in a anthocyanidin synthase (ans) gene found in the cells of the modified plant, and wherein the mutation results in reduced or eliminated expression of the anthocyanidin synthase (ans) gene.

17. A seed of the modified plant of claim 16.

18. The modified plant of claim 16, wherein the modified plant is a Medicago truncatula plant.

19. The modified plant of claim 16, wherein the plant is alfalfa, clover, soybean, grape, cacao, tea or strawberry plant.

20. A method for producing a modified plant having modified proanthocyanidin (PA) content in cells of the plant compared to an unmodified plant of the same species, comprising:
reducing or eliminating expression of the leucoanthocyanidin dioxygenase (ldox) gene in plant cells;
reducing or eliminating expression of the anthocyanidin synthase (ans) gene in the plant cells; and
using the plant cells to produce a modified plant having reduced or eliminated expression of the leucoanthocyanidin dioxygenase (ldox) gene and reduced or eliminated expression of the anthocyanidin synthase (ans) gene and modified proanthocyanidins (PAs) in cells of the modified plant, wherein the modified proanthocyanidins (PAs) comprise starter units consisting of catechin and extension units consisting of catechin.

21. The method of claim 20, wherein the step of reducing or eliminating expression of the leucoanthocyanidin dioxygenase (ldox) gene comprises introducing a mutation into a leucoanthocyanidin dioxygenase (ldox) gene in substantially all cells of the modified plant, wherein the mutation results in reduced or eliminated expression of the leucoanthocyanidin dioxygenase (ldox) gene in the modified plant.

22. The method of claim 20, wherein the step of reducing or eliminating expression of the anthocyanidin synthase (ans) gene comprises introducing a mutation into a anthocyanidin synthase (ans) gene in substantially all cells of the modified plant, wherein the mutation results in reduced or eliminated expression of the anthocyanidin synthase (ans) gene in the modified plant.

23. The method of claim 20, wherein the plant is a Medicago truncatula plant.

24. The method of claim 20, wherein the plant is alfalfa, clover, soybean, grape, cacao, tea or strawberry.

25. The method of claim 20, wherein the modified plant has increased soluble proanthocyanidin (PA) content compared to unmodified plants of the same species.

26. The method of claim 20, wherein the modified proanthocyanidins (PAs) have increased resistance to oxidation and reduced toxicity compared to proanthocyanidins in unmodified plants of the same species.

27. A modified plant having modified proanthocyanidins (PAs), wherein the modified proanthocyanidins (PAs) comprise starter units consisting of catechin and extension units consisting of catechin, wherein substantially all cells of the modified plant comprise a mutation in a leucoanthocyanidin dioxygenase (ldox) gene found in cells of the modified plant and a mutation in a anthocyanidin synthase (ans) gene found in the cells of the modified plant, and wherein the mutations result in reduced or eliminated expression of the leucoanthocyanidin dioxygenase (ldox) gene and the anthocyanidin synthase (ans) gene.

28. A seed of the modified plant of claim 27.

29. The modified plant of claim 27, wherein the modified plant is a Medicago truncatula plant.

30. The modified plant of claim 27, wherein the plant is alfalfa, clover, soybean, grape, cacao, tea or strawberry plant.


SEQUENCE LISTING (14 sequences)

SEQ ID NO: 1 - DNA, 22 bp, Artificial Sequence
  Primer Tnt1-F
acagtgctacctcctctggatg

SEQ ID NO: 2 - DNA, 21 bp, Artificial Sequence
  Primer ANS-Tn-F
atgggaacggtggctcaaaga

SEQ ID NO: 3 - DNA, 30 bp, Artificial Sequence
  Primer Tnt1-R
tgtagcaccgagatacggtaattaacaaga

SEQ ID NO: 4 - DNA, 22 bp, Artificial Sequence
  Primer LDOX-Tn-F
ggaagtcaaaagagtacaaact

SEQ ID NO: 5 - DNA, 26 bp, Artificial Sequence
  Primer ANS-F
atgggaacggtggctcaaagagttga

SEQ ID NO: 6 - DNA, 29 bp, Artificial Sequence
  Primer ANS-R
tcattttttgggatcatctttcttctcct

SEQ ID NO: 7 - DNA, 30 bp, Artificial Sequence
  Primer LDOX_F
atggaagtcaaaagagtacaaactttagct

SEQ ID NO: 8 - DNA, 26 bp, Artificial Sequence
  Primer LDOX_R
ctactggggaatcttgttgaatttgc

SEQ ID NO: 9 - DNA, 22 bp, Artificial Sequence
  Primer Tub-F
tttgctcctcttacatcccgtg

SEQ ID NO: 10 - DNA, 20 bp, Artificial Sequence
  Primer Tub-R
gccatggagagacctctagg

SEQ ID NO: 11 - DNA, 1071 bp, Medicago truncatula
atgggaacggtggctcaaagagttgaaagcttagccttgagtggtatatcatcaatcccaaaagaatatgtgagaccaaaagaagagttagcaaacataggtaacatctttgatgaagaaaaaaaagaaggtcctcaagttccaacaatagacctaaaagaaataaactcttcagatgaaatagttagaggaaaatgtagagagaagcttaagaaagctgcagaagaatggggtgtgatgcatttagtgaaccatggtatatctgatgatcttattaatcgtttgaagaaagctggtgaaacattttttgagcttcctgttgaagaaaaagagaaatatgcaaatgatcaaagttctgggaagattcaaggttatggaagtaaattagctaataatgctagtggtcaacttgaatgggaagattatttctttcattgcatttttccagaggataaacgtgatttgtctatatggcccaagacacctgctgattatactaaggttacaagcgaatatgcaaaggaattaagagtcctagctagcaagataatggaagtgttatctcttgaactagggttggaaggtggaaggttagaaaaagaagctggtggaatggaagagcttttacttcaaatgaaaattaattactacccaatttgccctcaaccagagctagcacttggagttgaagcccatacagatgtaagttcacttactttcctcctccataatatggtgccaggtttgcaacttttctatgagggcaaatgggtcactgcaaaatgtgtacctgattcaattctcatgcatattggtgacacaattgagatacttagcaatggaaagtacaaaagtatccttcatcgtggattggtgaataaggaaaaagttagaatatcttgggcagtgttttgtgaaccacctaaggagaaaattattctgaagccacttcctgaacttgtaactgaaaaagaaccagcaaggtttccacctcgcacttttgctcagcatattcatcacaaactttttaggaaggatgaggaggagaagaaagatgatcccaaaaaatga

SEQ ID NO: 12 - protein, 356 aa, Medicago truncatula
MGTVAQRVESLALSGISSIPKEYVRPKEELANIGNIFDEEKKEGPQVPTIDLKEINSSDEIVRGKCREKLKKAAEEWGVMHLVNHGISDDLINRLKKAGETFFELPVEEKEKYANDQSSGKIQGYGSKLANNASGQLEWEDYFFHCIFPEDKRDLSIWPKTPADYTKVTSEYAKELRVLASKIMEVLSLELGLEGGRLEKEAGGMEELLLQMKINYYPICPQPELALGVEAHTDVSSLTFLLHNMVPGLQLFYEGKWVTAKCVPDSILMHIGDTIEILSNGKYKSILHRGLVNKEKVRISWAVFCEPPKEKIILKPLPELVTEKEPARFPPRTFAQHIHHKLFRKDEEEKKDDPKK

SEQ ID NO: 13 - DNA, 996 bp, Medicago truncatula
atggaagtcaaaagagtacaaactttagcttgtaatcagctaaaggagcttccaccacaattcattcgcttagcaaatgaaaggccagagaatacaaaggccatggagggtgttactgtgcctatgatttcattgtctcaaccacataaccttttagtgaagaaaatcaatgaagctgcttctgagtggggtttctttgtgatcactgaccatggtatatctcaaaaacttattcaaagtttgcaagatgtgggccaggagtttttttctctccctcaaaaggagaaagagacatatgcaaatgatccatctagtggtaaatttgatggctatggaacaaagatgaccaagaaccttgaacaaaaggttgagtgggttgattattattttcatctcatgtctcctcattctaagttgaattttgagatgtggcccaaaagtcctccttcgtacagggaagtagtacaagaatacaataaagagatgttaagggtgacagacaatattttggagcttttgtctgaaggactagaattggagagtaagactttgaagtcttgtttgggaggtgaagaaatagaattagaaatgaagataaatatgtatccaccatgtccacaacctgaattggcattaggagttgagccccatactgatatgtctgccattacactacttgttccaaatgatgttccaggccttcaagtttggaaggacaataattgggttgcagtaaattacttgcaaaatgcactctttgtgcacattggtgaccaacttgaggtgttgagcaatgggaggtacaagagtgtcttgcacagaagcttggtgaacaaggaacgcaagcgtatgtcctgggcagtatttgttgctcctccacatgaggtcgtggttggacctcttcctccgctcgtcaatgatcaaaaccctgccaaattttcaacaaaaacctatgctgagtatcgctatcgcaaattcaacaagattccccagtag

SEQ ID NO: 14 - protein, 331 aa, Medicago truncatula
MEVKRVQTLACNQLKELPPQFIRLANERPENTKAMEGVTVPMISLSQPHNLLVKKINEAASEWGFFVITDHGISQKLIQSLQDVGQEFFSLPQKEKETYANDPSSGKFDGYGTKMTKNLEQKVEWVDYYFHLMSPHSKLNFEMWPKSPPSYREVVQEYNKEMLRVTDNILELLSEGLELESKTLKSCLGGEEIELEMKINMYPPCPQPELALGVEPHTDMSAITLLVPNDVPGLQVWKDNNWVAVNYLQNALFVHIGDQLEVLSNGRYKSVLHRSLVNKERKRMSWAVFVAPPHEVVVGPLPPLVNDQNPAKFSTKTYAEYRYRKFNKIPQ